(19) United States
(12) Patent Application Publication
(10) Pub. No.: US 2012/0282351 A1
(43) Pub. Date: Nov. 8, 2012

(54) System and Method for The Prevention of Bacterial and Fungal Infections Including Urinary Tract Infections (UTI) Using N-Halogenated Amino Acids

(75) Inventors: Ramin Najafi, Novato, CA (US); Mansour Bassiri, Novato, CA (US); Lu Wang, Moraga, CA (US); Behzad Khosrovi, El Cerrito, CA (US)

(73) Assignee: NovaBay Pharmaceuticals, Inc.

(21) Appl. No.: 13/523,676

(22) Filed: Jun. 14, 2012

Related U.S. Application Data
(62) Division of application No. 11/544,180, filed on Oct. 5, 2006.
Application No. 11/544,180 is a continuation of application No. 12/562,982, filed on Sep. 18, 2009, now abandoned.
(60) Provisional application No. 60/724,753, filed on Oct. 6, 2005.

(51) Int. Cl. A61K 31/185; A01N 59/00; A01N 41/04; A01P 3/00; A61P 31/12; A61P 31/10; A61K 33/00; A01P 1/00

(52) U.S. Cl. 424/661; 424/723; 514/578

(57) ABSTRACT

Disclosed is a system that prevents the development of infection and biofilm establishment in medical devices in general, and in particular Urinary Tract Infections (UTI), including Catheter-Associated Urinary Tract Infections (CAUTI). The system comprises a medical device (such as a catheter) and an antimicrobial composition containing an antimicrobial compound. A medical device delivers the composition both to the inside and/or outside portions of the device, as well as to the inside of the bladder itself and to the urethra. Reduction or elimination of the infection may be accomplished by irrigating the medical device, bathing the bladder, or irrigating the bladder with the composition.

### CROSS-REFERENCE TO RELATED APPLICATIONS

This application claims the benefit of U.S. Provisional Application No. 60/724,753, filed Oct. 6, 2005, the full content thereof fully incorporated herein by reference.

### SUMMARY OF THE INVENTION

This invention relates to a system that prevents the development of infection and biofilm establishment in medical devices in general, and in particular Urinary Tract Infections (UTI), including Catheter-Associated Urinary Tract Infections (CAUTI). The system comprises a medical device (such as a catheter) and an antimicrobial composition containing an antimicrobial compound. A medical device delivers the composition both to the inside and/or outside portions of the device, as well as to the inside of the bladder itself and to the urethra. Reduction or elimination of the infection may be accomplished by irrigating the medical device, bathing the bladder, or irrigating the bladder with the composition. Additionally, the medical device may be disinfected by such compositions prior to or during insertion through the urethral orifice. The medical device may also be stored in the compositions described herein. In addition to catheters or catheter-like devices other invasive medical devices such as pacemakers, heart valves, implantable devices, breast implants, intra-bone implants, stents, surgical plates, etc. may also be stored in the compositions described herein. The materials detailed in this invention include compositions comprising N-halogenated amino acids. The medical device may also be stored in the compositions described. The relevant compositions have broad-spectrum, non-specific, rapid antimicrobial activity and are effective against planktonic microorganisms, and microorganisms associated with biofilm and encrustation.

### BACKGROUND OF THE INVENTION

Over 40% of hospital acquired infections are Urinary Tract Infections (UTIs) and most of these are Catheter-Associated Urinary Tract Infections (CAUTIs), occurring in patients with urinary catheters (Hashmi, Kelly et al. 2003). In fact, urinary catheters are the second most common cause of bacteremia (Maki and Tambyah 2001). Bacteremia is the presence of viable bacteria in the circulating blood. Various approaches designed to prevent CAUTI are in use; however, even in combination, they may only delay the onset of CAUTI but remain unable to prevent it.

Bacteriuria (the presence of bacteria in normally sterile urine) develops in 5% of catheterized patients per day (3-10%); almost all catheterized patients have bacteriuria by 30 days. Since asymptomatic bacteriuria may not be diagnosed initially, 10-25% of patients with bacteriuria develop UTI (Saint and Chenoweth 2003).

In 1-4% of patients with bacteriuria, the infection spreads into the kidney or bloodstream, leading to potentially lethal bacteremia (viable bacteria in the blood) (Saint and Chenoweth 2003).

The main reason for bacterial growth leading to CAUTI and bacteremia is the establishment of biofilm on the surfaces of the catheter (Morris, Stickler et al. 1999; Maki and Tambyah 2001; Tenke, Riedl et al. 2004; Trautner and Darouiche 2004). Biofilm is a matrix produced and inhabited by bacteria that leads to the development of microbial colonies encased in an adhesive, usually polysaccharide, material that is attached to a surface (e.g. the device). In addition to providing a reservoir of bacteria, biofilm can also result in catheter encrustation by crystal deposits created by the bacteria that, over time, can restrict flow through the catheter or even block it completely.

In one aspect, the system of this invention is effective by: (a) impeding bacterial build-up and (b) killing bacteria in and around the medical device, bladder and urinary tract. Such bacteria build up in and around the medical device and the bladder may include planktonic bacteria or bacteria in the form of biofilm, such as bacteria embedded in biofilm. Planktonic bacteria are free-floating bacteria, as opposed to sessile bacteria in biofilms. The system is also useful in treating or preventing the formation of biofilm, killing bacteria embedded in biofilm, and removing biofilm. The system is also well tolerated, in particular, by inflamed or infected bladder tissue due to its low cytotoxicity. This unique combination of properties allows this system to effectively combat bacteriuria, thus limiting progression to CAUTI and bacteremia.

### FIELD OF THE INVENTION

The present invention relates to a system and methods for providing antimicrobial treatments. The system comprises a medical device (e.g. a catheter) and an antimicrobial composition. In a particular aspect, the methods comprise flushing, washing, instilling, irrigating and/or coating the medical device for the treatment, prevention or inhibition of infection by killing microbes and preventing microbial biofilm formation. The system may be provided in kits or trays for performing such treatment options.

The term “microbes” as used herein includes bacteria, fungi and viruses inhabiting areas in and around a medical device when used in patients.

The composition is useful in maintaining the medical device free from blockage and obstruction. The composition is also useful for treating, preventing and inhibiting infection including both inside and outside a patient's bladder. The medical device treated with a composition described herein is less likely to result in bacteriuria leading to urinary tract infection in patients receiving the medical device; one such device is a urinary catheter. Other non-limiting medical devices include intravascular catheters, such as cardiac catheters, central venous catheters, peritoneal dialysis catheters, dialysis shunts, such as hemodialysis shunts, endotracheal tubes, surgical drains, and device accessories, such as ports.

Methods of using the pharmaceutical composition of the invention in the management and maintenance of a medical device, such as a urethral catheter, are also disclosed in the present application.

### BACKGROUND OF THE INVENTION

A urinary catheter is a flexible tube system that is placed in the body to drain and collect urine from the bladder. Urinary catheters are used to drain the bladder during and after certain surgical procedures. Urinary catheters are also used to manage urinary incontinence and/or urinary retention in both men and women.

Depending on the underlying medical condition of the patient, a urinary catheter may be used (a) on an intermittent basis for just long enough to empty the bladder, (b) short term (hours or days, e.g. intra- and immediately post-operation), (c) longer term (few days to weeks, e.g. post-operation), or (d) continuous or chronic long term (30 days or more, e.g. spinal cord injuries (SCIs) and in Long Term Care Facilities (LTCFs)). An indwelling catheter that is left in place for a period of time is in general attached to a sterile container to collect the urine.

The most commonly used Foley indwelling catheter is a soft silicone or latex tube that is inserted into the bladder through the urethra to drain the urine, and is retained by a small balloon inflated with air or liquid. Urinary catheters come in a large variety of sizes, materials (latex, silicone, uncoated or coated with other materials such as silicone, hydrogel, antibacterial agents, etc.), and types (Foley catheter, straight catheter, Coude-tip catheter, etc.).

Catheters are generally placed into the bladder through the urethra, but in some cases, a suprapubic indwelling catheter is placed directly into the bladder through a surgically-prepared opening (stoma) in the abdomen above the pubic bone.

Catheter-Related Complications:

Complications of indwelling catheter use may include catheter encrustation and obstruction, bacteriuria, urinary tract and/or kidney infections, which in turn may proceed to blood infections (bacteremia or septicemia (blood poisoning or septic fever) or septic shock). Intermittent catheter use may also result in bacteriuria (presence of bacteria in the urine) and subsequent urinary tract infection. Catheter encrustation stems from an infection caused by bacteria that produce urease; the increased activity of the urease results in an increased local pH and the formation of calcium and magnesium phosphate crystals. These crystals encrust the catheter and can cause partial or total blockage through of the catheter lumen (Stickler, Young et al. 2003).

Definition of CAUTI:

Catheter associated urinary tract infection (CAUTI) is one of the most common nosocomial (hospital-acquired) infections in acute- and extended-care hospitals in the United Sates. It can affect the bladder and urethra, which are collectively known as the lower urinary tract.

The underlying cause of CAUTI is the formation of a pathogenic biofilm. Urease-producing bacteria colonize the catheter surface and create a biofilm community embedded in a polysaccharide matrix. The increased urease generates ammonia, which raises the pH of the biofilm and the urine; in this environment, hard crystals made of calcium and magnesium phosphate are formed and become embedded in the matrix (Stickler, Jones et al. 2003). There are few, if any, effective strategies to impede this process. Urethral catheters inevitably become colonized with attached microorganisms that are part of the biofilm community. Individuals develop bacteriuria at a rate of 3-10% per day; incidence reaches 100% in chronically catheterized individuals by 30 days (Trautner, Hull et al. 2005). The development of biofilm and crystalline encrustation of surfaces of urinary catheters has been demonstrated in a laboratory model using Proteus mirabilis (Stickler, Jones et al. 2003). Prophylactic bladder irrigation with antibiotics do not prevent colonization and lead to antibiotic resistance; prophylactic irrigation with hydrogen peroxide is also ineffective (Cravens and Zweig 2000).

Important routes of entry for bacteria into the bladder occur during the process of insertion of the catheter through the urethral orifice and by migration along the external surface of the catheter during movement of the catheter. Microorganisms found in urinary infections include Escherichia coli, enteric gram-negative rods such as Proteus, Enterobacter and Klebsiella species, gram-positive bacteria, increasingly Candida yeast strains, and some enteric organisms such as Providencia and Pseudomonas (Hashmi, Kelly et al. 2003).

### DESCRIPTION OF RELATED ART

An effective treatment of CAUTI must essentially succeed in three areas: preventing/treating the infection, helping the catheter to resist encrustation and blockage due to the infection, and penetrating/eradicating the biofilm that allows the infection to thrive. A review of the literature, as summarized below, shows that there is presently no antimicrobial agent that solves all of these problems efficiently (Trautner and Darouiche 2004). The dominant problem in the strategies that have been attempted thus far is that flora resistant to the antimicrobial agents eventually reappear.

At present, the most effective strategy used to minimize CAUTI is the use of closed drainage system; however, enhancements to this system are still needed to further minimize CAUTI. One such enhancement involves surface modification of the catheter material—that is, engineering the catheter material to make it inhospitable to CAUTI-causing bacteria. A review of catheters containing silver alloys in their matrix has shown they are only partially effective in reducing catheter-related bacteria (Saint and Chenoweth 2003). Urinary catheters impregnated with other antimicrobial agents have also been investigated to varying degree; devices with minocycline and rifampin (Darouiche, Smith et al. 1999), nitrofurazone (Maki and Tambyah 2001) and released gentamicin (Cho, Lee et al. 2001; Maki and Tambyah 2001) show some promise. However, with all of these agents, it is not clear whether prolonged use will result in the patient developing a resistance to the relevant bacteria (Saint and Chenoweth 2003). In fact, although some believe that surface modification shows more promise than instillation or irrigation (Tenke, Riedl et al. 2004), others believe that surface modification for preventing CAUTI has produced lackluster results at best (Trautner and Darouiche 2004).

That said, antimicrobial agents delivered systemically, instilled in the bladder, or used to irrigate the catheter have, thus far, shown to be ineffective for preventing CAUTI (Trautner and Darouiche 2004). A particular concern of catheter irrigation as a treatment for CAUTI is that for long-term catheterizations, the treatment will become ineffective because the bacteria and other flora that cause the CAUTI become resistant to said antimicrobial agent (Maki and Tambyah 2001; Saint and Chenoweth 2003; Trautner and Darouiche 2004). Studies using the antibiotic neomycin and independently the antimicrobial povidone-iodine for irrigation have shown no benefit for treating CAUTI (Hashmi, Kelly et al. 2003).

The use of bladder irrigation or instillation has been recommended to prevent debris and stone formation as well as infection (Galloway 1997). Urinary catheters, and Foley catheters in particular, are highly susceptible to encrustation and blockage from crystals generated by the local bacteria (Stickler, Young et al. 2003); the use of an antimicrobial solution to irrigate the catheter may have some success in preventing encrustation and blockage. Laboratory experiments using triclosan as the antimicrobial agent have show promise in preventing encrustation (Stickler, Jones et al. 2003); however, long term use of this agent in the body may result in the emergence of resistant bacteria. Similarly, although there has been some success using chlorhexidine solutions for this purpose (Baillie 1987; Pearman, Bailey et al. 1991), it is not practical for long term use because the bacteria develop resistance to the chlorhexidine (Baillie 1987). Additionally, breaking the closed drainage system of the catheter increases risk of infection and physical injury to the patient (Galloway 1997; Cravens and Zweig 2000).

Yet another consideration in using antimicrobial agents in urinary catheters is whether or not the agent will be able to penetrate and dislodge biofilm. The use of saline for irrigating catheters has little to no effect in reducing bacteriuria and dislodging biofilm (Muncie, Hoopes et al. 1989). Thus far, the use of antimicrobial agents (as ointments and lubricants, in collection bags, impregnated within the catheter material, and with bladder instillation or irrigation) has also resulted in a failure to treat biofilms (Donlan and Costerton 2002; Tenke, Riedl et al. 2004).

### BRIEF DESCRIPTION OF THE FIGURES

FIG. 1 is a schematic representation of a catheter during the process of being inverted into the bladder.

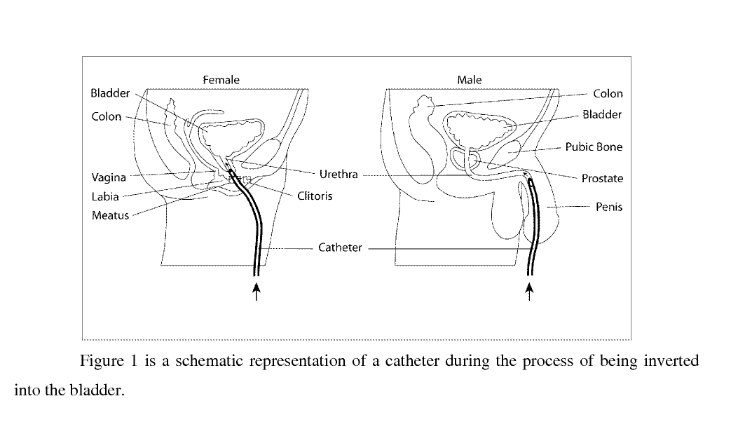

FIG. 2 is a schematic representation of a Foley catheter after insertion into the bladder, and the catheter is connected to a fluid container.

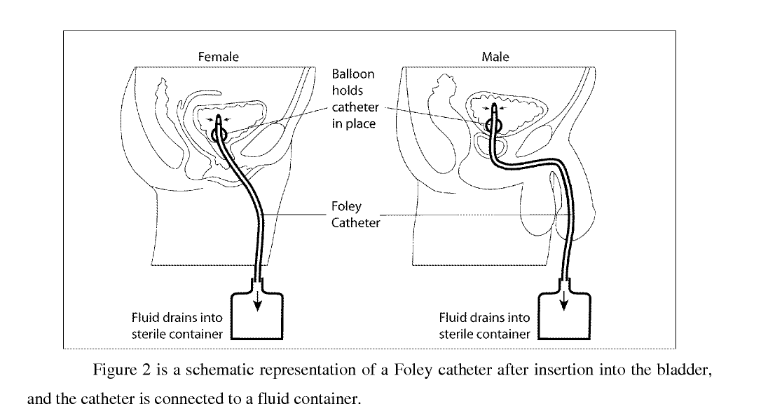

FIG. 3 is a schematic representation of a triple-lumen catheter after insertion into the bladder, wherein the catheter is connected to a reservoir and the fluid container.

FIG. 4 is a schematic representation of a test system for validation of a system for creating biofilm in vitro.

FIG. 5 is a graph of the results a biofilm prevention experiment.

### DESCRIPTION OF THE INVENTION

It is understood that any aspect or feature of the present invention whether characterized as preferred or not characterized as preferred may be combined with any other aspect or feature of the invention, whether such other feature is characterized as preferred or not characterized as preferred. For example, a feature described as preferred, for example a pH range, or a specific pH for a particular composition (for example, certain N-halogenated or N,N-dihalogenated amino acids of a specific formula) may be combined with another composition (N-halogenated or N,N-dihalohalogenated amino acids of another specific formula) without deviating from the present invention. This statement also applies to any combination of substituents. For example, a substituent characterized as preferred may be combined with any other substituent not characterized as preferred. The terms “include(s)” or “comprise(s)” are used as open terms interchangeably in the text of this specification.

The system provided herein comprises a medical device (such as a catheter but is not limited to a catheter) and an antimicrobial composition. The system provides alternative antimicrobial treatment options that do not have the undesirable properties of (a) inducing bacterial resistance and (b) significant toxicity. The antimicrobial composition comprises a compound that is an N-halogenated amino acid or derivative thereof, or a source of an N-halogenated amino acid, or mixtures thereof, or a combination of an N-halogenated amino acid and a hypohalous acid (HOHal, wherein Hal is chloro or bromo).

In one embodiment, there is provided a system wherein the medical device is a Central Venous Catheter (CVC). This type of catheter is placed into a large vein in the neck, chest, or groin. While all catheters can introduce bacteria into the bloodstream, CVCs can also cause Staphylococcus aureus sepsis and Staphylococcus epidermidis sepsis.

In one embodiment, there is provided a system wherein the medical device is a Peritoneal Dialysis Catheter. In case of kidney failure, peritoneal dialysis is used for removing waste such as urea and potassium from the blood, as well as removing excess fluid. Peritoneal dialysis requires access to the peritoneum, a natural semipermeable membrane surrounding the intestine. This access breaks normal skin barriers, and as people with renal failure generally have a slightly suppressed immune system, infection is a relatively common problem.

Peritoneal dialysis is typically done in the patient's home and workplace, but can be done almost anywhere; a clean area to work, a way to elevate the bag of dialysis fluid and a method of warming the fluid are all that is needed. The main consideration is the potential for infection with a catheter; peritonitis is a commonest serious complication, and infections of the catheter exit site or “tunnel” (path from the peritoneum to the exit site) are less serious but more frequent. Because of this, patients are advised to take a number of precautions against infection.

Peritoneal dialysis is a method for removing waste such as urea and potassium from the blood, as well as excess fluid, when the kidneys are incapable of this (i.e. in renal failure). It is a form of renal dialysis, and is thus a renal replacement therapy. Peritoneal dialysis works on the principle that the peritoneal membrane that surrounds the intestine, can act as a natural semipermeable membrane (see dialysis), and that if a specially formulated dialysis fluid is instilled around the membrane then dialysis can occur, by diffusion. Excess fluid can also be removed by osmosis, by altering the concentration of glucose in the fluid. Dialysis fluid is instilled via a peritoneal dialysis catheter, (the most common type is called a Tenckhoff Catheter) which is placed in the patient's abdomen, running from the peritoneum out to the surface, near the navel. Peritoneal dialysis catheters may also be tunnelled under the skin and exit alternate locations such as near the rib margin or sternum (called a presternal catheter), or even up near the clavicle. This is done as a short surgery. The exit site is chosen based on surgeon's or patient's preference and can be influenced by anatomy or hygeine issues. More details can be found in http://en.wikipedia.org/wiki/Peritoneal_dialysis or in Merck's Manual of Medical Information (hereinafter “MMOMI”), Home Edition, 1997, Editor-in-Chief Robert Berkow, M.D. pp. 600, 656-658.

In one embodiment, there is provided a system wherein the medical device is a Hemodialysis Shunt. The 3 most common types are an intravenous catheter, an arteriovenous (AV) Cimino fistula, or a synthetic graft. In all three cases, two tubes (or one tube with two lumen) are required to first remove blood to be cleansed and then to return clean blood to the body. Since hemodialysis requires continuous access to the circulatory system through the skin, patients undergoing hemodialysis have a portal of entry for microbes, which could lead to septicemia or an infection affecting the heart valves (endocarditis) or bone (osteomyelitis). More details can be found in Reference: http://en.wikipedia.org/wiki/Hemodialysis and MMOMI, pp. 654-657.

In one embodiment, there is provided a system wherein the medical device is an endotracheal tube (ETT). ETTs are put in the mouth and then down into the trachea (the airway) for the purpose of airway management and lung ventilation. These ETT's are at high risk for causing ventilator-associated pneumonia (VAP) in patients. VAP is a subset of hospital-acquired pneumonia and occurs after at least 48 hours of intubation and mechanical ventilation. There are several bacteria which are particularly important causes of VAP because of their resistance to commonly used antibiotics. More details can be found in Reference: http://en.wikipedia.org/wiki/Ventilator-associated_pneumonia.

In one embodiment, there is provided a system wherein the medical device is a surgical drain. A surgical drain is a tube used to remove pus, blood or other fluids from a wound or larger pleural effusions. Drains inserted after surgery help the wound to heal faster. Details can be found in MMOMI, pp. 225-227, 935-936 and 171.

In one embodiment, there is provided a system wherein the medical device is an accessory to a medical device susceptible to bacterial infection, such as a port.

The term “N-halogenated amino acid” in its broadest meaning includes halogenated amino acids in which at least one hydrogen of an amino group is replaced with halogen. The term also includes halogenated amino acids in which two hydrogen atoms of an amino group are replaced with two halogen atoms such as an “N,N-dihalogenated amino acid.” It further includes halogenated amino acids comprising at least two amino groups in which in more than one amino group hydrogen atoms may be replaced with halogen atoms.

Accordingly, in its broadest aspects the present invention provides an antimicrobial system, an antimicrobial composition or a method of treatment using the antimicrobial system or the antimicrobial composition. In one aspect, the antimicrobial composition comprises an N-halo- or N,N-dihaloamino acid of the formula (I))

A-C(R¹R^{o}R(CH₂)ₙ—C(YZ)—X′ (I)

or a derivative thereof; wherein A is hydrogen, HalNH— or Hal₂N— wherein Hal is halogen selected from the group consisting of chloro and bromo; R is a carbon carbon single bond or a divalent cycloalkylene radical with three to six carbon atoms, R¹ is hydrogen, lower alkyl or the group —COOH; R^{o} is hydrogen or lower alkyl; n is 0 or an integer from 1 to 13, or R¹ and R^{o} together with the carbon atom to which they attach form a (C₃-C₆)cycloalkyl ring; Y is hydrogen, lower alkyl or —NH₂, —NHHal or —NHal₂; and Z is hydrogen or lower alkyl; and X′ is hydrogen, —COOH, —CONH₂, —SO₃H, —SO₂NH₂, or —P(═O)(OH)₂. If R is a divalent cycloalkylene radical n will not exceed the integer 11. That is, n may be 0, 1, 2, 3, 4, 5, 6, 7, 8, 9, 10, or 11. That is, the amino acid including the acidic group X′ will have up to 16 chain atoms. In the divalent cycloalkylene radical or in the divalent radical —(CH₂)ₙ— one hydrogen may be substituted with —NHHal or —NHal₂. While the N-halo- or N,N-dihaloamino acids of the invention may contain up to 3 —NHHal or —NHal₂ groups, N-halogenated amino acids with 1 or 2 —NHHal or —NHal₂ groups are preferred. Most preferred are N,N-dihaloamino acids with 1 —NHal₂ group. This group may be in alpha-, beta-, gamma-, delta-, epsilon-, etc. to omega-position of the acidic groups R¹ (if R¹ is —COOH) or X′.

Derivatives of the compounds of formula (I) include pharmaceutically acceptable salts, esters with lower alkanols or esters containing an aryl group, lower alkanoyl derivatives of the —NH₂ group attached to the carbon atom to which the substituent X′ is attached. The term “lower” in this respect includes residues with 1 to 6, preferably 1 to 4 carbon atoms. The term “aryl” in this respect includes aryl with 5 to 10 carbon atoms in one or two aromatic rings, and optionally may include aliphatic side chains with 1 to 4 carbon atoms, and optionally may include up to two heteroatoms, such as N, O, or S in the ring system. Accordingly, as used herein, these ring systems comprising heteroatoms in the rings may be defined as “heteroaryls.” Aryl esters may be formed with compounds such as phenol, benzyl alcohol, alpha-naphthol, beta-naphthol, nicotinyl alcohol, etc.

In a preferred embodiment, R is a carbon carbon single bond and n is 0 or an integer from 1 to 7, more preferably 0 or an integer from 1 to 5, and most preferably 0 or an integer from 1 to 3, that is 1, 2 or 3. Also of interest are the N,N-dihalo amino acids in which n=4 or n=5 or n=6 or n=7 or n=8 or n=9. The N-halogenated amino acid can either be incorporated or embedded into the device material such that an N-halogenated amino acid is generated or activated on contact with moisture or aqueous fluids, such as bodily fluids. In another aspect, the antimicrobial compound may comprise an element of an aqueous solution and the solution may be used as part of the resulting antimicrobial composition.

As used herein, the term “N-halogenated amino acid” refers to an amino acid compound or composition wherein one or both of the hydrogen atoms on the amine group (—NH₂) of an amino acid compound may be replaced by a halogen, wherein the halogen or halo group is bromo or chloro.

Also provided are antimicrobial systems, compositions or methods which include an N,N-dihalo-amino acid of the formula (II))

Hal₂N—C(R¹R^{o})—(CH₂)ₙ—C(YZ)—X (II)

or a derivative thereof.

In the above formula, Hal is halogen selected from the group consisting of chloro and bromo; R¹ is hydrogen, lower alkyl or the group —COOH; R^{o} is hydrogen or lower alkyl, or R¹ and R^{o} together with the carbon atom to which they attach form a (C₃-C₆)cycloalkyl ring; n is 0 or an integer from 1 to 3; Y is hydrogen, lower alkyl, —NH₂, —NHHal or —NHal₂; and Z is hydrogen or lower alkyl; and X is —COOH, —CONH₂, —SO₃H or —SO₂NH₂. In one particular aspect of each of the above compositions, Hal is bromine or chlorine.

Derivatives of the compounds of formula (II) include pharmaceutically acceptable salts, esters with lower alkanols, esters containing an aryl group, lower alkanoyl derivatives of the —NH₂ group attached to the carbon atom to which the substituent X is attached. The term “lower” in this respect includes residues with 1 to 6, preferably 1 to 4 carbon atoms. The term “aryl” in this respect includes aryl with 5 to 10 carbon atoms in one or two aromatic rings, and optionally may include aliphatic side chains with 1 to 4 carbon atoms, and optionally may include up to two heteroatoms, such as N, O, or S in the ring system. Aryl esters may be formed with compounds such as phenol, benzyl alcohol, alpha-naphthol, beta-naphthol, nicotinyl alcohol, etc.

The systems, compositions and methods described herein also comprise N-monohalo amino acids of the formula)

HalNH—C(R¹R^{o})—(CH₂)ₙ—C(YZ)—X (IIA)

wherein Hal, R¹, R^{o}, n, Y, Z and X have the above-identified meanings; and their derivatives. Preferred are compounds of formula (IIA), wherein R¹ is lower alkyl or the group —COOH; R^{o} is lower alkyl, or R¹ and R^{o} together with the carbon atom to which they attach form a (C₃-C₆)cycloalkyl ring; and their derivatives. In one particular aspect of each of the above compositions, Hal is bromine or chlorine.

Derivatives of the compounds of formula (IIA) include pharmaceutically acceptable salts, esters with lower alkanols, esters containing an aryl group, lower alkanoyl derivatives of the —NH₂ group attached to the carbon atom to which the substituent X is attached. The term “lower” in this respect includes residues with 1 to 6, preferably 1 to 4 carbon atoms. The term “aryl” in this respect includes aryl with 5 to 10 carbon atoms in one or two aromatic rings, and optionally may include aliphatic side chains with 1 to 4 carbon atoms, and optionally may include up to two heteroatoms, such as N, O, or S in the ring system. Aryl esters may be formed with compounds such as phenol, benzyl alcohol, alpha-naphthol, beta-naphthol, nicotinyl alcohol, etc.

The present invention provides systems, compositions and methods which comprise an N,N-dihaloamino acid of the formula (III)

A-C(R¹R²)R(CH₂)ₙ—C(YZ)—X′ (III)

or a derivative thereof; where A is hydrogen or Hal₂N— wherein Hal is halogen selected from the group consisting of chloro and bromo; R is a carbon carbon single bond or a divalent (C₃-C₆)cycloalkylene radical with three to six carbon atoms, R¹ is hydrogen, lower alkyl or the group —COOH; R² is lower alkyl or R¹ and R² together with the carbon atom to which they attach form a (C₃-C₆)cycloalkyl ring; n is 0 or an integer from 1 to 13; Y is hydrogen, lower alkyl or —NH₂, —NHHal or —NHal₂; and Z is hydrogen or lower alkyl; and X′ is hydrogen, —COOH, —CONH₂, —SO₃H, —SO₂NH₂, or —P(═O)(OH)₂. If R is a divalent (C₃-C₆)cycloalkylene radical n will not exceed the integer 11. In other words the amino acid including the acidic group X′ will have up to 16 chain atoms. Optionally, in the divalent (C₃-C₆)cycloalkylene radical or the divalent radical —(CH₂)ₙ—, one hydrogen may be substituted with —NHHal or —NHal₂. While the N,N-dihaloamino acids of the invention may contain up to 3 —NHal₂ groups, N,N-dihaloamino acids with 1 or 2 —NHal₂ groups are preferred. Most preferred are N,N-dihaloamino acids with 1 —NHal₂ group. This group may be in alpha-, beta-, gamma-, delta-, epsilon-, etc. to omega-position of the groups R¹ or the groups R¹ (if R¹ is —COOH) or X′. Also included are N-monohalo amino, in particular N-monochloro amino acids and their derivatives wherein the —NHal₂ group of formula (III) is replaced with an —NHHal group [formula (IIIA)].

Derivatives of the compounds of formula (III), (IVA) or (IVB) (described below) include pharmaceutically acceptable salts, esters with lower alkanols, esters containing an aryl group, lower alkanoyl derivatives of the —NH₂ group attached to the carbon atom to which the substituent X or X′ is attached, and their N-monohalo amino acid derivatives. The term “lower” in this respect includes residues with 1 to 6, preferably 1 to 4 carbon atoms. The term “aryl” in this respect includes aryl with 5 to 10 carbon atoms in one or two aromatic rings, and optionally may include aliphatic side chains with 1 to 4 carbon atoms, and optionally may include up to two heteroatoms, such as N, O, or S in the ring system. Aryl esters may be formed with compounds such as phenol, benzyl alcohol, alpha-naphthol, beta-naphthol, nicotinyl alcohol, etc.

In a preferred embodiment R is a carbon carbon single bond and n is 0 or an integer from 1 to 7, more preferably 0 or an integer from 1 to 5, and most preferably 0 or an integer from 1 to 3.

In another aspect, a system, composition, and method with antimicrobial activity is provided comprising an N,N-dihaloamino acid of the formula (IVA) or a N-monohalo derivative thereof; (IVB)

Hal₂N—C(R¹R²)—(CH₂)ₙ—C(YZ)—X (IVA)

HalHN—C(R¹R²)—(CH₂)ₙ—C(YZ)—X (IVB)

wherein Hal is halogen selected from the group consisting of chloro and bromo; R¹ is hydrogen, lower alkyl or the group —COOH; R² is lower alkyl or R¹ and R² together with the carbon atom to which they attach form a (C₃-C₆)cycloalkyl ring; n is 0 or an integer from 1 to 3; Y is hydrogen, lower alkyl or —NH₂; and Z is hydrogen or lower alkyl; and X is —COOH, —CONH₂, —SO₃H or —SO₂NH₂; said derivative being selected from the group consisting of pharmaceutically acceptable salts, esters with lower alkanols, esters containing an aryl group, and lower alkanoyl derivatives of the —NH₂ group attached to the carbon atom to which the substituent X is attached. The term “aryl” in this respect includes aryl with 5 to 10 carbon atoms in one or two aromatic rings, and optionally may include aliphatic side chains with 1 to 4 carbon atoms, and optionally may include up to two heteroatoms, such as N, O, or S in the ring system. Aryl esters may be formed with compounds such as phenol, benzyl alcohol, alpha-naphthol, beta-naphthol, nicotinyl alcohol, etc.

In another aspect, the above-described composition comprising an N,N-dihaloamino acid of the formula (IVA) or a N-monohalo derivative thereof (IVB) is one in which R¹ is hydrogen, or lower alkyl; n is 0, 1 or 2; Y is hydrogen or lower alkyl; and X is —SO₃H or —SO₂NH₂; or a derivative thereof; said derivative being selected from the group consisting of pharmaceutically acceptable salts or esters with lower alkanols.

In a further aspect, the above-described compositions comprising an N,N-dihaloamino acid of the formula (IVA) or a N-monohalo derivative thereof (IVB) are those wherein Y and Z are hydrogen; X is —SO₃H; the derivative being selected from the group consisting of pharmaceutically acceptable salts. In another aspect of the above formula, Hal is chloro.

The pharmaceutically acceptable salts of compounds of formula (I), (II), (IIA), (III), (IIIA), (IVA) or (IVB) or their derivatives include salts with pharmaceutically acceptable cations. The salts of the N-halo- or N,N-dihaloamino acid includes salts of bases with the —COOH, —CONH₂, —SO₃H or —SO₂NH₂ groups. Pharmaceutically acceptable salts also include ammonium, alkali metal, magnesium, or calcium salts and any organic amine salts. Alkali metal salts, magnesium, calcium and aluminum salts are of interest. The alkali metal salts are of particular interest, particularly lithium, sodium, or potassium salts. In general, the salts of the halogenated amino acids may function as sources of the free halogenated amino acid which may be released when in contact with bodily fluids or when contacted with an acidic medium.

Examples of acid addition salts include, but are not limited to, mineral or organic acid salts of basic residues such as amines; alkali or organic salts of acidic residues such as carboxylic acids, and the like. Pharmaceutically acceptable salts include, but are not limited to, hydrohalides, sulfates, methosulfates, methanesulfates, toluenesulfonates, nitrates, phosphates, maleates, acetates, lactates and the like.

Lists of suitable salts are found in Remington's Pharmaceutical Sciences, 17th ed., Mack Publishing Company, Easton, Pa., 1985, p. 1418 or The Merck Index, Thirteenth Edition, 2001, Published by Merck Research Laboratories Division of Merck & Co., Inc. on pages MISC-22 and MISC-23, the disclosures of which are hereby incorporated by reference in their entirety. The pharmaceutically acceptable acid addition salts of the —NH₂ group attached to the carbon atom to which substituent X or X′ is attached include salts, among others, with hydrochloric acid, sulfonic acid, phosphoric acid, nitric acid, benzenesulfonic acid, toluenesulfonic acid, methanesulfonic acid, camphorsulfonic acid and other acids.

Further derivatives of the compounds of formulae (I), (II), (IIA), (III), (IIIA), (IVA) and (IVB) include esters of the groups —COOH or —SO₃H with lower alkanols, esters containing an aryl group (as herein described) and also lower alkanoyl derivatives of the amino group attached to the carbon atom to which substituent X or X′ is attached. Further derivatives of the compounds of formulae (I), (II), (IIa), (III), (IIIA), (IVA) and (IVB) also include the N-halo amino acids or the N,N-dihalo-amino acids in which certain groups of the amino acid molecule are protected by protecting groups. “Protecting group” as used herein, means a chemical group that (a) preserves a reactive group from participating in an undesirable chemical reaction; and (b) can be easily removed after protection of the reactive group is no longer required. Removal of the protecting groups may be performed by chemical synthesis or where desired, the protecting group may be removed when contacted with the appropriate condition or medium, such as with moisture or fluids, in vivo.

“Amino-protecting group” means a protecting group that preserves a reactive amino group that otherwise would be modified by certain chemical reactions. Non-limiting examples of amino protecting groups include the formyl group or lower alkanoyl groups with 2 to 4 carbon atoms, in particular the acetyl or propionyl group, the trityl or substituted trityl groups, such as the monomethoxytrityl group, dimethoxytrityl groups such as the 4,4′-dimethoxytrityl or 4,4′-dimethoxytriphenylmethyl group, the trifluoroacetyl, and the N-(9-fluorenyl-methoxycarbonyl) or “FMOC” group, the allyloxycarbonyl group or other protecting groups derived from halocarbonates such as (C₆-C₁₂)aryl lower alkyl carbonates (such as the N-benzyloxycarbonyl group derived from benzylchlorocarbonate), such as the benzyloxycarbonyl (CBZ group), or derived from biphenylalkyl halo carbonates, or tertiary alkyl halo carbonates, such as tertiary-butylhalocarbonates, in particular tertiary butylchloro-carbonate, or di(lower)alkyldicarbonates, in particular di(t-butyl)-dicarbonate, and the phthalyl group. Examples of other suitable protecting groups may be found in T. W. Greene, Protecting Groups in Organic Synthesis, 3rd edition, John Wiley & Sons, Inc. 1999. Halogenated amino acids in which certain functional groups (such as the amino or carboxy group) are protected may function as sources of unprotected or free halogenated amino acids when in contact with an acidic medium, water, or aqueous fluids, such as bodily fluids.

In the —CONH₂ or the —SO₂NH₂ group of the compounds of the formula (I), (II), (IIA), (III), (IIIA), (IVA) or (IVB) one or two hydrogen atoms may be substituted with one or two Hal atoms, wherein Hal is chloro or bromo, for example, resulting in compounds with —CONHCl, —CONCl₂, —SO₂NHBr or —SO₂NBr₂ groups. Similarly, in the alkylated or acylated —CONH₂ or the —SO₂NH₂ group, that are —CONHAlk or —CONHAc or the —SO₂NHAlk or —SO₂NHAc groups, wherein Alk is lower alkyl and Ac is lower acyl, the —NH hydrogen atom may be replaced with chloro or bromo. Lower alkyl and lower acyl mean groups with 1 to 4 carbon atoms.

The preferred derivatives are pharmaceutically acceptable salts.

In another aspect, the above-described compositions include the following compounds or a derivative thereof; the derivative being selected from the group consisting of pharmaceutically acceptable salts and esters with lower alkanols:

N,N-dichloro-2,2-dimethyltaurine; N-chloro-2,2-dimethyltaurine; N,N-dichloro-1,1,2,2-tetramethyltaurine; N-chloro-1,1,2,2-tetramethyltaurine; N,N-dibromo-2,2-dimethyltaurine; N-bromo-2,2-dimethyltaurine; N,N-dibromo-1,1,2,2-tetramethyltaurine; N-bromo-1,1,2,2-tetramethyltaurine; N,N-dichloro-2-methyltaurine; N-chloro-2-methyltaurine; N,N-dichloro-2,2,3,3-tetramethyl-β-alanine; N,N-chloro-2,2,3,3-tetramethyl-β-alanine; N,N-dichloro-3,3-dimethylhomotaurine; N-chloro-3,3-dimethylhomotaurine; N,N-dichloro2-methyl-2-amino-ethanesulfonic acid; N-chloro-2-methyl-2-amino-ethanesulfonic acid; N,N-dichloro-1-methyl-ethanesulfonic acid; N,N-dichloro-1-methyl-ethanesulfonic acid; N-chloroaminotrimethylene phosphonic acid; N,N-dibromo-2-amino-5-phosphonopantanoic acid; N-bromo 2-amino-5-phosphonopantanoic acid; N,N-dichloro aminoethylphosponic acid diesters, such as the diethylester; N-chloro aminoethylphosponic acid diesters, such as the diethylester; N,N-dichloro 1-amino-1-methylethane phosphonic acid; N-chloro 1-amino-1-methylethane phosphonic acid; N,N-dichloro 1-amino-2-methylethane phosphonic acid; N-chloro 1-amino-2-methylethane phosphonic acid; N,N-dichloro 1-amino-2-methylpropane phosphonic acid; N-chloro 1-amino-2-methylpropane phosphonic acid; N,N-dichloro leucine phosphonic acid; N-chloro leucine phosphonic acid; N,N-dichloro 4-amino-4-phosphonobutyric acid; N-chloro 4-amino-4-phosphonobutyric acid; (±) N,N-dichloro 2-amino-5-phosphonovaleric acid; (±) N-chloro 2-amino-5-phosphonovaleric acid; N,N-dichloro (+)2-amino-5-phosphonovaleric acid; N-chloro (+)2-amino-5-phosphonovaleric acid; N,N-dichloro d,1-2-amino-3-phosphonopropionic acid; N-chloro d,1-2-amino-3-phosphonopropionic acid; N,N-dichloro 2-amino-8-phosphonooctanoic acid; N-chloro 2-amino-8-phosphonooctanoic acid; N,N-dichloro leucine boronic acid; N-chloro leucine boronic acid; N,N-dichloro-β-alanine boronic acid; or N-chloro-β-alanine boronic acid; or a pharmaceutically acceptable salt or ester thereof.

In another aspect, the compositions described herein comprising a mono- or dihaloamino acid of the formula (I), (II), (IIA), (III), (IIIA), (IVA) or (IVB) or their derivatives are ones in which Hal is chloro. In yet another aspect, the compositions described herein comprising a mono- or dihaloamino acid of the formula (I), (II), (IIA), (III), (IIIA), (IVA) or (IVB) or their derivatives are ones in which Hal is bromo or chloro.

Further details of the N-halogenated amino acids and their derivatives and preferred N-halogenated amino acids, processes for the preparation of the N-halogenated amino acids are disclosed in pending PCT/US Serial No. PCT/US2006/002875, filed Jan. 25, 2006, the disclosure of which is incorporated by reference in its entirety. Preferred are N-halogenated amino acids for use in the antimicrobial system disclosed herein that are indicated as preferred in the above referenced pending application, and the preferences are incorporated by reference herein.

Further antimicrobial systems, compositions and methods comprise an N,N-dihaloamino acid of the formula (I)

A-C(R¹R^{o})R(CH₂)ₙ—C(YZ)—X′ (I)

or a derivative thereof; wherein A is hydrogen, HalHN— or Hal₂N—; Hal is halogen selected from the group consisting of chloro or bromo; but chloro is preferred; R is a carbon carbon single bond or a divalent cycloalkylene radical with three to six carbon atoms; R¹ is hydrogen, lower alkyl or the group —COOH; R^{o} is hydrogen or lower alkyl; or R¹ and R^{o} together with the carbon atom to which they attach form a (C₃-C₆)cycloalkyl ring; n is 0 or an integer from 1 to 5; Y is hydrogen, lower alkyl, —NH₂, —NHHal or —NHal₂; Z is hydrogen or lower alkyl; and X′ is hydrogen, —COOH, —SO₃H, or —P(═O)(OH)₂; if R is a divalent cycloalkylene radical and n is an integer from 1 to and including 3, the divalent radical R or divalent radical —(CH₂)ₙ— being optionally substituted with —NHHal or —NHal₂; the derivative being a pharmaceutically acceptable salt, ester with lower alkanols, or lower alkanoyl derivative of the —NH₂ group attached to the carbon atom to which the substituent X′ is attached.

In another aspect of the above formula, R^{o} is lower alkyl. In yet another aspect of the above formula, R is a carbon carbon single bond.

In another aspect, there is provided a system, composition or method wherein the N-halo- or N,N-dihaloamino acid comprises 1 or 2 —NHHal or —NHal₂ groups, or wherein the N-halo- or N,N-dihaloamino acid comprises 1 —NHHal or —NHal₂ group. In one variation of the above, the —NHHal or —NHal₂ group is in the alpha, beta or gamma position to the group X′. In another aspect, A is —NHHal or —NHal₂. In yet another aspect, the —NHHal or —NHal₂ group is attached to the divalent radicals R or —(CH₂)ₙ—. In another aspect of the above, Hal is chloro.

In one aspect of the invention, the derivative is a pharmaceutically acceptable salt.

In another aspect of the invention, there is provided a system, composition and method which comprise an N-halogenated amino acid with the formula (II))

Hal₂N—C(R¹R^{o})—(CH₂)ₙ—C(YZ)—X (II)

or wherein the Hal₂N— group is replaced with the HalHN— group [formula (IIA)], or a derivative thereof; wherein Hal is halogen selected from the group consisting of chloro or bromo; R¹ is hydrogen, lower alkyl or the group —COOH; R^{o} is hydrogen or lower alkyl; or R¹ and R^{o} together with the carbon atom to which they attach form a (C₃-C₆)cycloalkyl ring; n is 0 or an integer from 1 to 3; Y is hydrogen, lower alkyl or —NH₂; Z is hydrogen or lower alkyl; and X is —COOH, —SO₃H; said derivative being selected from the group consisting of pharmaceutically acceptable salts, esters with lower alkanols, and lower alkanoyl derivatives of the —NH₂ group attached to the carbon atom to which the substituent X is attached.

In yet another aspect, there is provided a system, composition and method which comprise an N-halogenated amino acid with formula (II))

Hal₂N—C(R¹R^{o})—(CH₂)ₙ—C(YZ)—X (II)

or wherein the Hal₂N— group is replaced with the HalHN— group [formula (IIA)], or a derivative thereof; wherein Hal is halogen selected from the group consisting of chloro or bromo; R¹ is hydrogen, lower alkyl or the group —COOH; R^{o} is hydrogen or lower alkyl; n is 0 or an integer from 1 to 3; Y is hydrogen, lower alkyl or —NH₂; Z is hydrogen or lower alkyl; and X is —COOH, —CONH₂, —SO₃H or —SO₂NH₂; the derivative being selected from the group consisting of pharmaceutically acceptable salts, esters with lower alkanols, and lower alkanoyl derivatives of the —NH₂ group attached to the carbon atom to which the substituent X is attached. The Hal₂N— group in formula (II) may be replaced with the HalHN— group [formula (IIA)].

In one aspect, there is provided a system, composition and method which comprise an N-halogenated amino acid with the formula (IVA) or (IVB)

Hal₂N—C(R¹R²)—(CH₂)ₙ—C(YZ)—X (IVA)

HalHN—C(R¹R²)—(CH₂)ₙ—C(YZ)—X (IVB)

or a derivative thereof; wherein Hal is halogen selected from the group consisting of chloro or bromo; R¹ is hydrogen, lower alkyl or the group —COOH; R² is lower alkyl; n is 0 or an integer from 1 to 3; Y is hydrogen, lower alkyl or —NH₂; Z is hydrogen or lower alkyl; and X is —COOH, —SO₃H or —SO₂NH₂; the derivative being selected from the group consisting of pharmaceutically acceptable salts, esters with lower alkanols, and lower alkanoyl derivatives of the —NH₂ group attached to the carbon atom to which the substituent X is attached. In one variation, R¹ is hydrogen, or lower alkyl; n is 0, 1 or 2; Y is hydrogen or lower alkyl; Z is hydrogen or lower alkyl; and X is —SO₃H or —SO₂NH₂; or a derivative thereof; the derivative being selected from the group consisting of pharmaceutically acceptable salts or esters with lower alkanols. In another variation, Y and Z are both hydrogen; X is —SO₃H; or the derivative is a pharmaceutically acceptable salt.

In another variation, the invention provides an antimicrobial system, compositions and methods which comprise the use of a halogenated amino acid of the formulae (I), (II), (IIA), (III), (IIIA), (IVA) or (IVB) or of a derivative thereof, or of a specific halogenated amino acid, or of a source of a halogenated amino acid in conjunction with the use of a hypohalous acid or a hypohalous acid source as described in copending PCT/US Serial No. PCT/US2006/002875, filed Jan. 25, 2006, the disclosure of which is incorporated by reference in its entirety. If in the antimicrobial system, composition or method an N-halogenated source is used in conjunction with a hypohalous source, it is preferred that the halogen atom(s) in the halogenated amino acid and the halogen atom in the hypohalous acid are the same. That is, an N-chlorinated amino acid will be used together with hypochlorous acid. For example, N,N-dichloro-2,2-dimethyl taurine may be used together with hypochlorous acid. If an amino-protected halogenated amino acid is used in conjunction with hypochlorous acid, the unprotected halogenated amino acid may be released in the antimicrobial system, compositions and methods disclosed herein. This means that the presence of hypohalous acid in the composition results in the removal of the protecting group and in the release of the free N-halogenated amino acid.

The use of the antimicrobial compounds described herein may be useful as being an effective treatment of Urinary Tract Infection (UTI) and in particular of Catheter-Associated Urinary Tract Infection (CAUTI) in these critical areas: minimizing the opportunity for bacterial biofilm formations that would allow the infection to thrive and potentially cause bacteriuria in catheterized patients, penetrating/eradicating or reducing the biofilm that is able to form, and helping the catheter to resist encrustation and blockage due to the infection and subsequent biofilm formation.

The system may also be useful in treating and preventing other microbial infections in conjunction with other devices described herein, such as viral, yeast or fungal infections, in particular those associated with bacterial infections. The antimicrobial compounds, systems and treatments described herein may also be of use to reduce the risk of bacteremia or septicemia (blood poisoning or septic fever) or septic shock, particularly in immuno-compromised patients, for example elderly patients, patients that are undergoing chemotherapy, or patients affected by viral diseases, such as HIV patients, patients receiving transplants whose immune system may be down regulated by medications and also in general, patients subject to invasive procedures.

The antimicrobial compound employed in the practice of the present invention is one that is not classified as an antibiotic. For purposes of the present invention, the term “antibiotic” is defined as a chemical substance produced by microorganisms, or synthetic or semi-synthetic analog, or a derivative of such a chemical substance that can inhibit or destroy susceptible microorganisms (e.g. penicillin).

It is an object of the present invention to avoid the overuse of these traditional antibiotics, although they may, if desired, be used systemically in conjunction with the system of the invention. Compositions of antimicrobial compounds and antimicrobial compositions are provided for use in flushing and coating medical devices, especially catheters and ports.

The preferred medical devices of this invention are urinary catheters as described herein.

Urinary catheters consist of a tube that is inserted through the urethra into the bladder. In men, it is inserted through the tip of the penis, and in women, it is inserted through the meatus.

The best known catheter is the double-lumen Foley catheter, a device often employed with hospital patients recovering from surgery. The tip of the Foley catheter is inserted until it enters the bladder. An inflatable, small, bi-lateral balloon near the tip holds the catheter in place when inflated. The tip of the tube has openings to allow flow of urine into a container for collection. A side port, for example a “T” junction, may be introduced into the catheter pathway in order to facilitate repeated instillation and irrigation while minimizing avenues for added infection. These catheters can be flushed using intermittent back flow (that is, irrigation of the treatment composition from the port opening back up the catheter into the bladder).

In cases where the need for flushing or rinsing of the bladder is anticipated, for example to remove blood and debris after surgery, a triple-lumen Foley catheter may be used instead. This catheter-type has an additional lumen through which fluid from a reservoir can be provided into the bladder and flushed out through the main lumen together with urine into a container. These catheters can be flushed using continuous flow.

Typical catheters used in accordance with the treatment described herein are disclosed in U.S. Pat. No. 4,245,639 and U.S. Pat. No. 4,337,775. These catheters have drainage means (for example, a cannula) and means for holding the drainage means in place in the bladder of a patient (for example, an inflatable balloon). The drainage and holding means have inner and outer surfaces that may be exposed to bacterial biofilm formation.

Catheters are generally placed into the bladder through the urethra, but in some cases, a suprapubic indwelling catheter is placed directly into the bladder through a surgically prepared opening (stoma) in the abdomen above the pubic bone.

The Antimicrobial Composition:

In one aspect of the invention, there is provided a method for treating a medical device and/or surrounding tissue with a biocidally (i.e. ability of inactivating pathogens) effective amount of an antimicrobial composition. Amounts are given in mM, which equals millimoles per liter. In another aspect, there is provided a method of treating, inhibiting, reducing or preventing infection associated with the use of the medical device before or after it has been inserted in a patient.

The method comprises treating or contacting the medical device with a biocidally-effective amount of an aqueous antimicrobial composition, the composition comprising:

(A) an antimicrobial compound, comprising
(1) at least one halogenated amino acid or a halogenated amino acid source, optionally in combination with a hypohalous acid (HOHal, wherein Hal is chloro or bromo) or hypohalous acid source;
(2) the halogenated amino acid being at least one N-halogenated or N,N-dihalogenated amino acid, alone or in combination;
(3) the halogenated amino acid concentration ranging from about 1 mM to about 1000 mM in the composition; and

(B) an aqueous solution, comprising
(1) at least one saline component (halide salt) selected from the group consisting of sodium chloride, sodium bromide, potassium chloride, potassium bromide, magnesium chloride and magnesium bromide;
(2) the pH of the composition ranging from about 2 to about 8, preferably 2.6 to 7.5;
(3) the saline component (halide salt) concentration ranging from 0 to about 20 g/L, preferably about 2 to about 20 g/L of the aqueous composition, more preferably about 4 to about 12g/L; and optionally
(4) other constituents including acids, buffering and chelating agents, either organic or inorganic.

The pH range of choice depends on the condition to be treated, the elements and constituents comprising the composition and their relative ratios, the preferred pH ranges for the compounds used, as well as other variables employed for the particular compositions and their method of use.

In some aspects of the invention the pH range for compositions containing N-halo amino acid compounds may be between pH 2 and pH 8. In certain variations, the pH ranges of the composition may be between pH 4 and pH 6, a range from pH 4.5 to pH 5.5, a range from pH 2 to pH 4, a range from pH 2.5 to pH 3.5, a range from pH 5 to pH 8, a range from pH 6 to pH 7, or as desired to optimize the biological activity of the composition.

For compositions containing mono-halo amino acid (or N-halo amino acid) compounds, the pH range is preferably between pH 7 and pH 8. In certain condition, it has been observed that these compounds undergo increasing disproportionation reactions at lower pH values into the free amine and the corresponding di-chloro compound.

For composition comprising the use of HOBr or HOCl, the pH of the composition is preferably between pH 3.5 and pH 7.5, or between 4 to pH 7, between pH 5 to pH 6.

Accordingly, depending on the particular therapeutic applications, the particular conditions, the particular elements comprising the compositions, the pH of the composition comprising the above compounds or mixtures of the above compounds, the pH range should be chosen accordingly to optimize the effectiveness of the composition as determined by one skilled in the art.

As noted herein, the halide salt is an optional component of the composition. That is, the halide salt may be present in the composition or the halide salt may be absent. In one particular aspect of the composition, the halide salt may be present in the composition at a concentration of about 0.05 g/L or more.

In another aspect, the method comprises administering the above aqueous solution to the patient using the medical device. In one particular variation, the medical device is a catheter.

An N-halogenated amino acid source is a composition that has the ability to release an N-halogenated amino acid depending on its environment. It may be a physical composition, for example, a carrier of an N-halogenated amino acid that is compatible with the N-halogenated amino acid and not oxidizable by the N-halogenated amino acid or not oxidizable by the combination of halogenated amino acid with the hypohalous acid if such a combination is employed. Such a carrier may be a non-oxidizable material, such as a cloth that may be used in conjunction with the system described herein, for example for the purpose of cleaning urethral openings. Another N-halogenated amino acid source may include non-oxidizable microcapsules that will release an N-halogenated amino acid when in contact with water or aqueous systems or solutions, such as a bodily fluid. Another N-halogenated amino acid source may be an N-halogenated amino acid precursor or prodrug which releases an N-halogenated amino acid when contacted with water or aqueous systems or solutions, such as a bodily fluid. The preferred N-halogenated source is an N-halogenated amino acid and most preferably N,N-dichloro-2,2-dimethyl taurine.

In another aspect, this disclosure describes an antimicrobial composition for use with medical devices as discussed herein.

A preferred device treatment or medical treatment of a patient uses an antimicrobial composition containing the antimicrobial compound N,N-dichloro 2,2-dimethyl taurine. Other preferred N-halogenated compounds are described above and are comprised by formula (I), (II), (IIA), (III), (IIIA), (IVA) or (IVB) or identified by specific chemical names above.

In general, in the systems described herein the constituent 5 and/or each constituent of constituent 6 of claim 1 below may be present in concentrations of 0, or about 1 mM to about 100 mM.

Preferred devices or treatments comprise N-halogenated amino acid concentrations up to 500 mM, or up to 300 mM, or up to 200 mM, or up to 150 mM. Devices or treatments are more preferred where the N-halogenated amino acid concentration or the combination of N-halogenated amino acid with a hypohalous acid concentration (hereinafter “combination concentration”) ranges from about 4 mM to about 100 mM in the composition. In one variation, the N-halogenated amino acid concentration or combination concentration ranges from about 10 mM to about 70 mM, or about 5 mM to about 40 mM. In another variation, the N-halogenated amino acid concentration or combination concentration ranges from about 50 mM to about 80 mM, or about 4 mM to about 50 mM. In another variation, the N-halogenated amino acid concentration or combination concentration ranges from about 60 mM to about 75 mM, or about 30 mM to about 500 mM. In another variation, the total or combination concentration (N-halogenated amino acid and hypohalous acid concentration) may be between 2 mM to 20 mM.

Concerning the saline component (halide salt), the preferred inorganic salt is sodium chloride at a concentration of up to about 2% by weight, preferably about 0.2 to about 2% by weight, more preferably 0.4 to about 1.2% by weight NaCl which is about four-tenth to slightly higher than normal or isotonic saline solution. The term “halide salt” and the term “saline component” are used interchangeably herein to reflect the fact that the compositions described herein aim to achieve biologically or physiologically acceptable salt concentrations. In yet another aspect of the invention, the inorganic salt in the aqueous solution is at a concentration of about 0.7 to about 1.0 by weight %. In a variation of the above, the inorganic salt is sodium chloride. If an N-bromo or N,N-dibromo amino acid is used, it is preferred that no saline component is present, because the N-bromo- or N,N-dibromo amino acid would decompose. According to Parker's McGraw-Hill Dictionary of Scientific and Technical Terms, S. P. Parker, editor, Fifth Edition, “normal saline”, “physiological saline”, “physiological salt solution” are defined as a “solution of sodium chloride in purified water, containing 0.9 grams of sodium chloride in 100 milliliters; isotonic with body fluids.” For different halide salts such as lithium halides, potassium halides, and the like, the concentration of the salt in making up an isotonic solution may differ from the concentration of sodium chloride in an aqueous solution in order to maintain the desired osmolarity of the solution of the invention.

More effective devices may be treated with a composition where the saline component concentration ranges from about 7 to about 10 g/L of the composition. Likewise, in the most effective antimicrobial treatment options, patients are treated with antimicrobial compositions where the halide salt concentration is about 7 to about 10 g/L, with 9 g/L being most preferred.

The preferred pH for the treatment ranges from about 3.3 to about 5.5, and even more preferred, from about 3.5 to about 5.0. Depending on its use the pH may be from about 3.5 to about 4.5, or about 3.5 to about 4.0, 3.8 to about 4.3; 4.0 to about 4.5; 4.3 to about 4.8 or at about 4.5 to about 5.0, or at about 4.8 to about 5.3, or at about 5.0 to about 5.5. The pH may be at any pH range within the broad pH range from about 2.0 to about 8.0, for example, 2.6 to 6; 6 to 8, etc. For example, for patients with the risk of encrustation forming around the tip of the catheter more acidic pH ranges would be preferred to counteract crystal deposits from calcium or magnesium phosphate crystals.

As disclosed above, bacteria in areas in and around a medical device or the bladder produce urease, an enzyme which hydrolyzes urea to carbon dioxide and two equivalents of ammonia. The hydrolysis raises the pH of the urine. As a result of the increased pH, the formation of calcium and magnesium phosphate deposits is favored, which may result in encrustation of the tip of a catheter.

Buffer Systems: To counteract the increase of the pH, appropriate buffer systems may be used to maintain the pH at a lower range. The selection of the optimum buffer systems and buffer conditions and buffer concentrations is known to a person skilled in the art. It may among other factors, depend on the pH of the urine, the amount of urea in the urine, the degree and kind of bacterial infection, etc. However, in general, buffer amounts may be present in the antimicrobial compositions herein described in an amount to maintain the pH in and around the catheter and the bladder of the patient between 3 and 6, or 3.5 to 5.

Examples of buffer systems comprising electrolyte solutions include well known buffer systems such as Clark and Lubs solutions, pH 2.2-4.0 (Bower and Bates, J. Res Natn. Bur. Stand. 55, 197 (1955)); beta,beta-dimethylglutaric acid-NaOH buffer solutions, pH 3.2-7.0 (Stafford, Watson, and Rand, B B A 18, 318 (1955)); sodium acetate-acetic acid buffer solutions, pH 3.7-5.6; succinic acid-NaOH buffer solutions, pH 3.8-6.0 (Gomeri, Meth. Enzymol. 1, 141 (1955)); sodium cacodylate-HCl buffer solutions, pH 5.0-7.0 (Pumel, Bull. Soc. Chim. Biol. 30, 129 (1948)); Na₂HPO₄—NaH₂PO₄ buffer solutions, pH 5.8-7.0 (Gomeri and Sorensons, Meth. Enzmol. 1, 143 (1955)); potassium biphthalate/HCl, pH 3.0 to 3.8; potassium biphthalate/NaOH pH 4.0-6; KH₂PO₄/NaOH, pH 6.0-7.0; and monopotassium phosphate/NaOH, pH 6.0 to pH 8.0 or NaOH/boric acid, pH 7.8 to pH 8.0 (see OECD Guideline for Testing Chemicals “Hydrolysis as a Function of pH,” Adopted 12 May 1981, 111, pp. 10-11). With regard to the stability of the N-halo amino acids, a higher pH range would be preferred, because at a lower pH the N-halo amino acid would disproportionate to the N,N-dihalo amino acid and the des-halogenated amino acid. Considering stability alone, the preferred pH range for the N-halo amino acid would be from about 7 to about 8. However, the disproportionation reaction would not interfere with the use of the N-halo amino acid within the systems, compositions and uses disclosed herein, because the N,N-dihalo amino acids have a stronger antimicrobial effect than the corresponding N-halo amino acids. However, it would be a consideration for the preparation of kits and trays where longer stability would be required. As concerns the N,N-dihalo amino acids, the pH range is not as critical, because these compounds are stable over a broader pH range.

Acids, Esters and Salts: A preferred acid is one that is at a biologically safe concentration and is biologically compatible with the antimicrobial compound. The acid is a member of the group selected from acetic acid, benzoic acid, propionic acid, oxalic acid, hydrochloric acid, phosphoric acid, sulfuric acid, boric acid, diethylenetriamine pentaacetic acid, and esters of p-hydroxybenzoic acid (Parabens), or the biologically acceptable salt form of the acid is a member of the group selected from potassium citrate, potassium metaphosphate, sodium acetate, and sodium phosphate.

Chelating Agents: The antimicrobial composition may also comprise a biologically acceptable, and in the presence of the antimicrobial compound, stable chelating agent that prevents encrustation of the device (e.g. by insoluble salts of Ca²+ or Mg²+). Other examples include malic acid and maltol.

Depending on the nature of the constituents, each of these constituents may serve multiple functions. For example, a single constituent may have acidic, buffering and/or chelating properties. The preferred concentration ranges for other constituents is 1 to 100 mM. The chelating agent concentration may be selected that the chelating agent chelates up to about 10 mM, up to about 5 mM, up to about 2 mM or up to about 1 mM of a member selected from the group consisting of calcium, magnesium and mixtures thereof.

The buffering agent may be selected to achieve any desired pH or pH range for the system and compositions described herein For example, for a particular system the buffering agent composition is selected to maintain the pH between about 3.5 to about 4.5.

Because the catheter surface plays an important role in biofilm formation, preferred device surfaces have increased hydrophilicity which provide a softer surface for tissue contact and reduced susceptibility of CAUTI and bacteriuria. Increased surface hydrophilicity may be affected by hydrogel-coating, for example, with polyvinyl pyrrolidone and polyethylene glycol.

Alternatively, the antimicrobial compound (i.e., the N-halogenated amino acid source) can either be incorporated or embedded into the device material such that the N-halogenated amino acid is generated or activated on contact with aqueous fluids. Furthermore, the compound may be allowed to slowly diffuse into the surrounding space. Alternatively, it could be present in an inactive state and be activated by a chemical reaction with a substrate that it supplied to the catheter in an aqueous solution.

Optionally, a patient may be treated systemically with broad spectrum or specific antibiotics at the same time, in combination with the methods of the present invention.

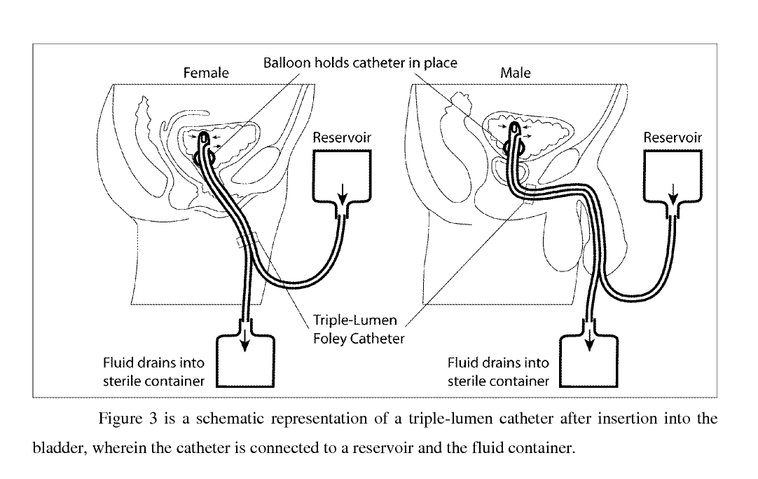

In some instances the device comprises the antimicrobial composition contained in a reservoir connected with the device (see FIG. 3). Commonly the reservoir is elevated above the position of the device itself, for example a hanging bottle.

The reservoir may be attached to the catheter device through a dispensing conduit or device which may have flexible or rigid tubing. The device may be configured in a way wherein the reservoir is configured with an antimicrobial composition dispensing device in a drainage receptacle receiving a biological fluid. The drainage receptacle may be configured in such a way that multiple dispensing devices could be placed into the drainage receptacle, such as when emptying the urine from the receptacle. Preferred devices have the dispensing device in the lower portion of the drainage receptacle and the antimicrobial composition may be dispensed from the dispensing device into the receptacle. The reservoir may also be configured to secure the catheter in place when the device is inserted into the bladder of a patient.

The uses of catheters that benefit most from the treatment described herein are the uses of indwelling catheters, for example, a Foley catheter. Alternatively, the catheter may also be an intermittent catheter.

Likewise, patients that benefit from the treatment described herein are patients that are suffering from infections that may be both related and unrelated to the use of catheters. Examples include interstitial cystitis caused or aggravated by bacterial infections, or fungal cystitis, underactive bladder diseases, particularly caused by neurological injuries or disorders, overactive bladder diseases, lack of bladder control, such as urinary incontinence patients, patients suffering from CAUTI, bacteriuria, or urethral injuries, etc.

The devices herein described may be treated with an above-described antimicrobial composition prior to insertion through the urethral orifice. Some device treatment options include irrigation, flushing, rinsing or washing of the device. Some treatment options include irrigation and instillation using the compositions described herein into a patient's bladder.

Procedures for the Method of Treatment:

The method of treating, inhibiting, reducing or preventing infection in or near a medical device before or after the device has been inserted in a patient, and the method of preventing or treating infection in a patient after the device has been inserted in a patient comprises the following individual treatment steps in isolation or in combination:

(a) contacting the device with the above defined antimicrobial composition prior to insertion in a patient or after removal from a patient;

(b) washing, bathing or flushing the device with the above defined antimicrobial composition prior to insertion in a patient or after removal from a patient;

(c) irrigating the device with above defined antimicrobial composition after insertion in a patient, to remove encrustations on the device; or

(d) instilling through the device an antimicrobial composition into the bladder of a patient to treat or prevent a fungal, viral or bacterial infection of the lining of the bladder or urethra.

The above individual treatment steps are described below.

The treatment of a patient to treat, inhibit or prevent microbial infection should use a sufficient amount of a solution comprising a composition as described herein. A sufficient amount means a dose range between 1 and 100 ml for instillation and 10 to 1,000 ml for irrigation with a N-halogenated amino acid concentration or combination concentration as described herein for one treatment procedure (for example, irrigation or instillation), or as deemed necessary for the particular application. It is self-evident that in case of severe infection the procedure may have to be repeated to maximize the antimicrobial effect.

The present invention also relates to a device treated with the above described antimicrobial composition or a method of treating, inhibiting, reducing or preventing infection in or near a medical device before or after the device has been inserted in a patient comprising (a) treating or contacting the device, or the patient through the device, with a biocidally effective amount of the above described antimicrobial composition, or (b) administering to the device or to the patient through the device the above described antimicrobial composition. In another aspect, the present invention also relates to a method of treating, inhibiting, reducing or preventing infection in or near a medical device before or after said device has been inserted in a patient or a method of treating, inhibiting or preventing infection in a patient comprising (a) treating or contacting the device, or the patient through the device, with a biocidally effective amount of the above described antimicrobial composition, or (b) administering to the device or to the patient through the device the above described antimicrobial composition.

The amount of solution of the antimicrobial composition used for the treatment of a catheter device should be enough to fill the device. Such devices typically have internal volumes in the range of about 1 to 3 mL. However, the volume will, of course, vary with the length and diameter of the tubing of the device, which may depend on the individual patient. Larger volumes (e.g. 20-100 ml) of the antimicrobial composition as described herein may be needed for procedures such as bladder instillation.

Pre-Treatment Using the Antimicrobial Composition:

Although the medical treatment options described herein and the treated devices of the present invention are primarily concerned with introducing the antimicrobial compositions into catheters that are already in place, those skilled in the art will appreciate that contacting the patient's body at and around the site of insertion can aid in the elimination of sites for bacterial growth. Thus, patients can be treated and the surfaces of medical devices, such as catheters, can be pre-treated by the compositions of the present invention to prevent bacteriuria and thereby prevent the infection that may ensue. In one method, the medical device can be treated with a composition initially and then, after insertion, with repeated periodic antimicrobial treatment options described above.

Packaging:

The invention also relates to kits or trays that include the above described antimicrobial compositions that are useful for the treatment methods described herein. For example, such kits or trays may comprise a closed sterile catheter syringe pre-filled with the antimicrobial composition for catheter insertion, irrigation or instillation purposes. The trays or kits may include lubricant, prepackaged disinfectant supplies, additional prepackaged antimicrobial composition, pre-packaged alcohol wipes etc. In addition, the kits or trays may contain instructions how to use the kits or trays in the treatments described herein.

Microorganisms Treated:

Use of catheters treated with the antimicrobial compositions described herein reduces bacteriuria caused by, but not limited to, the following microorganisms (bacteria, viral and fungi): Staphylococcus aureus, Staphylococcus saprophyticus, Staphylococcus epidermidis, and other Staphylococcus species, Escherichia coli, Pseudomonas aeruginosa, Proteus mirabilis, Providencia stuartii, Pseudomonas sp. Enterococci, Proteus species, Klebsiella pneumoniae, Enterobacter species, Candida species, Candida galabrata, Candida albicans, Serratia marcescens, Citrobacter spp., Morganella morganii, Enterococcus faecalis, Stenotrophomonas species, Clostridium difficile, Lactobacillus species, and other uropathogenic microorganisms, adenovirus and herpes.

Treatment of a medical device, such as catheter, or antimicrobial treatment of a patient in accordance with this disclosure, includes treatment of the device, such as a catheter with the above described antimicrobial composition or administering such compositions to a patient through a catheter device. Such treatments include treatment of the catheter prior to its use and also treatment of the catheter while inserted in a male or female patient, adult, or child. The treatment includes any form of contact of a composition described herein with the catheter and antimicrobial treatment with the compositions described herein through administration to a patient. Non-limiting examples of such treatments include rinsing, washing, flushing, instillation and irrigation. The treatment also includes inflation of the balloon of a catheter with the antimicrobial composition. The treatment may also include bathing a patient's bladder with the compositions described herein.

### EXAMPLE 1

### Representative Compositions for Use with a Catheter Include

### Composition A:

33 mM N,N-dichloro 2,2-dimethyl taurine
0.9% NaCl
pH 4

### Composition B

33 mM N,N-dichloro 2,2-dimethyl taurine
0.4% NaCl
pH 4

### Composition C:

33 mM N,N-dichloro 2,2-dimethyl taurine
0.9% NaCl
pH 3.5

### Composition D:

20 mM N,N-dichloro 2,2-dimethyl taurine
0.9% NaCl
pH 2

### Composition E:

50 mM N,N-dichloro 2,2-dimethyl taurine
0.9% NaCl
pH 5
15 mM malic acid

### Composition F:

100 mM N,N-dichloro 2,2-dimethyl taurine
0.9% NaCl
pH 7
20 mM phosphate
20 mM malic acid

### Composition G

100 mM N-chlorotaurine
0.9% NaCl
20 mM total sodium phosphate buffer
pH 7.5

### Composition H

40 mM N,N-dichlorotaurine
20 mM HOCl
0.9% NaCl
10 mM acetic acid-sodium acetate (pH 4)

### Composition I

40 mM N,N-dichloro 2,2-dimethyl taurine
40 mM N-chloro 2,2-dimethyltaurine
100 mM sodium phosphate buffer (pH 7.5)
0.9% NaCl

Compositions A-I are prepared in the form of solutions. All solutions are made from purified water. The N-halogenated amino acids may be used after having been prepared by halogenation of the amino acid in aqueous solution in situ. If N-chloro 2,2-dimethyltaurine or N-chlorotaurine is used in situ to prepare a solution, then the in situ preparation will have to be made at a pH above 8 to ensure that only the mono-halogenated compound is formed. After completion of the halogenation, a buffer may be used to adjust the pH within the range from 7 to 8, and saline may be added. For the preparation of the N,N-dihalo amino acid containing compositions the exact pH is not as critical, provided the pH is adjusted to 6 or below 6. Alternatively, some of the compositions can be made from the N-halogenated amino acids in solid form which are dissolved in purified water and the buffer and the saline component may be added. This is the method of choice if a composition with a low saline concentration is to be prepared.

### EXAMPLE 2

### Inserting a Catheter Through the Urethra in Women and Men

The following is a description of a general procedure for inserting a catheter and for using the antimicrobial composition. Assuming that a person skilled in the art is proficient in sterile techniques and in working with catheters, including dealing with obstructions and knowing when to call a physician, nurse or medical specialist for assistance, only those steps relevant to this invention are described. The other steps of the procedure (for example, hand cleansing or sanitization, lubrication of the catheter, inflating the balloon of the catheter once the catheter is in place), safeguards (for example, the use of sterile gloves and how to use them), instructions to the treated patient (for example, breathing or relaxation instructions) are familiar to physicians or nurses.

Use 5-100 ml of the antimicrobial Composition C (as described in Example 1) to clean the urethral opening.

Throughout the process of insertion, gently push antimicrobial composition up through the catheter, so that the urethra gets disinfected prior to coming into contact with the catheter.

Use 1-20 ml of the antimicrobial Composition C to wet the catheter as it is being inserted through the urethra into the bladder.

### EXAMPLE 3

### Opening a Partially Obstructed (Encrusted) Urinary Catheter

The following is an example of a catheter irrigation procedure to improve flow through a partially obstructed catheter. The catheter is irrigated with the composition to remove an encrustation at the tip of the catheter (plug) so that the urine can drain from the bladder.

Irrigation of a catheter in accordance with the invention may constitute a procedure to open a plugged urinary catheter with the above described antimicrobial composition. Assuming that a person skilled in the art is proficient in sterile technique and in working with catheters, including dealing with obstructions and knowing when to call a physician, nurse or medical specialist for assistance, only those steps relevant to this invention are described. The other steps of the procedure (how to deflate the balloon), safeguards (for example, the use of sterile gloves and how to use them) instructions to the treated patient (for example, breathing or relaxation instructions) are familiar to physicians or nurses.

The following instructions can be used for an irrigation procedure with the composition disclosed herein:

Draw up 1 to 100 mL of the antimicrobial Composition A (as described in Example 1) into a syringe.

After disconnecting the catheter from the drainage tubing, insert the syringe with the antimicrobial composition into the catheter.

Gently push on the plunger of the syringe to slowly push the composition into the catheter. Do not force the composition into the catheter.

If the composition does not flow easily into the catheter, gently pull back on the plunger to aspirate (withdraw) fluid, using very little force.

After inserting the antimicrobial composition into the catheter, remove the syringe from the catheter and insert the connecting tubing.

Check the tubing after reconnecting to see if urine is flowing. If no urine is flowing after 10 to 15 minutes, repeat the irrigation process.

### EXAMPLE 4

### Bladder Instillation Procedure

The following instructions can be used for an instillation procedure with the composition disclosed herein for a patient. Assuming that a person skilled in the art is proficient in sterile technique and in working with catheters, including dealing with obstructions, only those steps relevant to this invention are described. The other steps of the procedure, safeguards (for example, the use of sterile gloves and how to use them) instructions to the treated patient (for example, breathing or relaxation instructions) are familiar to physicians or nurses.

Bladder instillation, also called bladder wash or bath, may help relieve inflammation, infection or repair the bladder's protective lining. During this treatment, the bladder is filled with the antimicrobial composition B described herein using a catheter. The composition is held inside the bladder for a period of time ranging from 15-30 minutes. Then the composition is urinated through the urethra or drained from the bladder through the catheter. Instillation treatments may be repeated several times over a period of two to three months. Instillation of 20 to 80 mL of the composition described herein directly into the bladder may be accomplished by an aseptic syringe and allowed to remain inside the bladder for 10 to 100 minutes. The antimicrobial composition may be expelled by spontaneous voiding. It is recommended that the treatment may be repeated every week until maximum symptomatic relief is obtained. Thereafter, time intervals between treatments may be increased appropriately.

### EXAMPLE 5

### Efficacy of the Antimicrobial Composition

We have devised a dynamic in vitro model using traditional microbiological methods to assess the antimicrobial efficacy of 33 mM N,N-dichloro 2,2-dimethly taurine in 0.9% saline at pH 3.5, as compared to physiological saline in disinfecting intra-luminal and extra-luminal indwelling Foley catheter.

The effectiveness of this antimicrobial composition on E. coli or Pr. mirabilis biofilm covered Foley catheter has been demonstrated using the materials and methods detailed below:

### Materials:

Foley Catheter, manufactured by BARD
N,N-dichloro 2,2-dimethyl taurine (33 mM) in 0.9% saline pH 3.5
Escherichia coli ATCC 25922
Proteus mirabilis ATCC 29245

Neutralizer Broth containing: A broth containing dextrose, lecithin, sodium thiosulfate, pancreatic digest of casein, Tween® 80, yeast extract, sodium bisulfate, sodium thioglycollate, monopotassium phosphate, and bromcresol purple.

Nutrient Broth and agar

Spectrophotometer

The ability of N,N-dichloro 2,2-dimethyl taurine to destroy biofilm formation was evaluated as follows. First, biofilm was established on 1-cm-long pieces of catheter for 48 hours in nutrient broth in the presence of either Proteus mirabilis or Escherichia coli. Subsequently, the biofilm-bearing pieces of catheter were exposed to 33 mM N,N-dichloro 2,2-dimethyl taurine in 0.9% saline at pH 3.5 over various periods of time. After the exposure, the pieces of catheter were transferred into 1 mL of neutralizer broth to stop the reaction. 0.1 mL (10%) of the neutralizer broth was then plated out onto nutrient agar and the number of colonies was counted. The CFU (Colony Forming Unit) values obtained were multiplied by 10 to obtain the actual CFU/mL values per treated sample.

In order to measure the amount of live bacteria left on the pieces of catheter following treatment, the biofilm-bearing pieces of catheter were transferred into tubes containing fresh growth medium. After allowing growth in a shaker at 37° C. for 4 hours, Optical Density (OD) was read at 600 nm

Results are shown in the tables below. Cases where data were not collected are indicated by n.d.

Escherichia coli 25922

| | N,N-dichloro 2,2-dimethyl | Saline |
|---|---|---|
| Duration of | taurine 33 mM | 0.9% |
| exposure | pH 3.5 | pH 3.5 |
| Minutes | CFU/mL | CFU/mL |
| <1 | 0 | >>3000 |
| 5 | 0 | n.d. |
| 10 | 0 | n.d. |
| 20 | 0 | n.d. |
| 30 | 0 | n.d. |
| 45 | 0 | n.d. |
| 60 | 0 | n.d. |
| 120 | 0 | >>3000 |

Results: E. coli infected sample but untreated had CFU/ml=>>3000 colonies and OD₆₀₀=0.60

Proteus Mirabilis 29245

| | N,N-dichloro 2,2-dimethyl | Saline |
|---|---|---|
| Duration of | taurine 33 mM | 0.9% |
| exposure | pH 3.5 | pH 3.5 |
| Minutes | CFU/mL | CFU/mL |
| <1 | >>3000 | >>3000 |
| 5 | 1210 | n.d. |
| 10 | 150 | n.d. |
| 20 | 150 | n.d. |
| 30 | 100 | n.d. |
| 45 | 20 | n.d. |
| 60 | 10 | n.d. |
| 120 | 260 | >>3000 |

Results: Pr. mirabilis infected but untreated had CFU/ml=>>3000 colonies and OD₆₀₀=0.17

Under the conditions of this study, Foley catheters infected with E. coli and Pr. mirabilis for 48 hours and then treated with 33 mM N,N-dichloro 2,2-dimethyl taurine in 0.9% saline at pH 3.5 were shown to have minimal recoverable CFU/mL bacteria. This was also shown by very low optical density readings (average of 0.057 OD₆₀₀ units, individual data not listed) following the attempt to re-culture the bacteria from treated catheters. By contrast, the same infected catheter treated with physiological saline resulted in no suppression, but rather significant re-growth of bacteria even as long as 120 minutes of treatment (both by viable count and by optical density). Therefore, the in vitro biofilm disinfection model described here demonstrated significant antimicrobial properties for 33 mM N,N-dichloro 2,2-dimethyl taurine in 0.9% saline at pH 3.5, as compared to physiological saline.

Visual examination showed build up of biofilm on the catheter surface during infection and its subsequent removal by 33 mM N,N-dichloro 2,2-dimethyl taurine in 0.9% saline at pH 3.5, but not by saline.

### EXAMPLE 6

### Establishes an In Vitro Model for Biofilm Eradication and Prevention by N,N-dichloro 2,2-dimethyl taurine (DCDMT)

Part A: Setup and Validation of System for Creating Biofilm In-Vitro

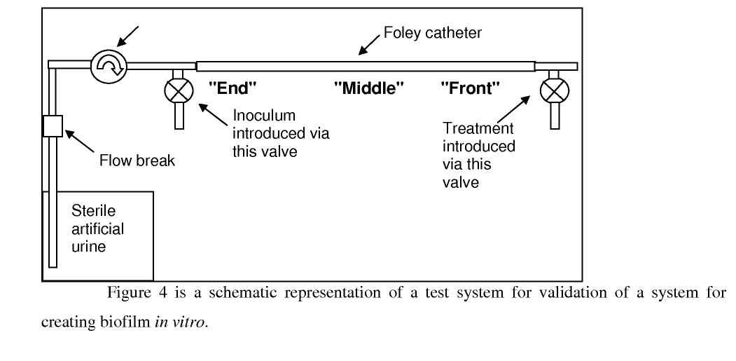

A test system was established which utilized size 14 Foley catheters (supplied by NovaCal), which were cut and installed into a pre-sterilized flow system (FIG. 4) using aseptic techniques. The system consists of five parallel channels, one channel per catheter. Sterile medium was supplied to the system via a flow-break, to prevent back-growth into the medium reservoir. The entire system was placed in a 37° C. incubator. After conditioning the system with artificial urine medium for 30 minutes, 2.0 ml inoculum from an overnight culture of urease positive Escherichia coli ATCC 25922 grown in artificial urine medium at 37° C. was introduced into the system via the valve closest to the flow break (bladder side of catheter). Each inoculum was tested to confirm urease production. After inoculation, the system remained under static conditions (no flow) for two hours, to allow for bacterial attachment to the catheters. Flow of artificial urine medium was then initiated and maintained at a rate of 0.75 ml/min for 3 days.

Initial experiments were conducted to evaluate consistency of biofilm formation in the model system. Viable cell counts indicated that by Day 3 biofilm was established at 10⁸ CFU/cm² throughout the length of the catheter. Day 5 and Day 7 counts remained at approximately that level. It was decided that treatment would be performed on Day 3 to prevent the possibility of biofilm detachment occurring.

Part B: Biofilm Eradication by N,N-dichloro 2,2-dimethyl taurine

Test Articles Used Were:
Sterile saline
N,N-dichloro 2,2-dimethyl taurine, 4 mM, pH 4, 0.9% by weight NaCl
N,N-dichloro 2,2-dimethyl taurine, 40 mM, pH 4, 0.9% by weight NaCl

To Demonstrate Treatment Efficacy:

20 ml of each treatment solution N,N-dichloro 2,2-dimethyl taurine and sterile control solution were loaded into 30 ml syringes and connected to a syringe pump. Sterile sections of tubing were attached from the syringe to the valve furthest from the flow break (bag end of the catheter). This end is designated as FRONT for sampling purposes. The pump was turned on and the treatments were introduced at 2.0 ml/min for 10 minutes through the catheters. Excess medium and treatment solution was captured in a waste container. After 10 minutes, the syringe pump was turned off and the solutions were left stationary in the catheters for 30 minutes. The solutions were then withdrawn back through the catheter into the syringe, medium flow was resumed for a 30 minute rinse time and the catheters were then sampled.

For Efficient Sampling:

Each catheter was divided into 3 segments (front, middle, end) and each segment was subsampled. One subsample was used to determine bacterial populations by plate count, another subsample was analyzed by staining with the LIVE/DEAD® Baclight™ bacterial viability kit (L7012, Molecular Probes, Oregon, USA) using confocal laser microscopy (CSLM), the third sample was imaged using scanning electron microscopy (SEM).

For viable cell counts, a 3.0 centimeter section of tubing was removed and scraped with a sterile stainless steel rod (using aseptic technique) into a tube containing 10.0 ml of sterile phosphate-buffered saline (PBS). The tubes were then sonicated for two minutes and the suspension was vortexed for one minute. The number of viable (culturable) bacteria was enumerated by serial dilution in PBS and plate counts using the spread-plate technique. Results were expressed as CFU/cm² and are calculated as follows:

$$\frac{( Mean C F U )}{( Volume Plated )} \times Dilution \times \frac{( Volume scraped into )}{( Surface Area )}$$

The surface area of the internal lumen of the catheter section scraped was determined to be 2.826 cm².

Results and Data Interpretation:

Three treatment runs were performed on 3 catheters each: One treated with PBS or sterile 0.9% saline as control, one treated with 4 mM and one treated with 40 mM N,N-dichloro 2,2-dimethyl taurine. The results are shown in Table 2. The Log (CFU/cm²) was calculated from the average of 9 treatments, consisting of 3 treatments of 3 catheter pieces for each Test Article.

TABLE 2
Biofilm eradication experiment. Results indicate NovaCal's treatments
is effective at removing biofilm cells from contaminated catheters in a
model urinary catheter system.
Average Log CFU/cm² on catheter section after treatment

| | | | | average | Log |
|---|---|---|---|---|---|
| | front | middle | end | 3 pieces | reduction |
| PBS or Saline | 9.0 | 9.1 | 9.1 | 9.1 | n.a. |
| N,N-dichloro 2,2-dimethyl | 4.1 | 3.9 | 5.1 | 4.4 | 4.7 |
| taurine [4 mM] | | | | | |
| N,N-dichloro 2,2-dimethyl | 3.7 | 3.8 | 3.8 | 3.8 | 5.3 |
| taurine [40 mM] | | | | | |

The Urinary Catheter Model developed at the CBE (Center for Biofilm Engineering at Montana State University hap://www.erc.montana.edu) was shown to be an effective urinary catheter model test system. E. coli biofilms grew to uniform viable cell counts at approximately 10⁸ cfu/cm² in 3 days. This uniformity of biofilms grown in the five test catheters within the model allowed for the comparison of biofilms exposed to different treatment conditions in the catheters.

NovaCal's product, N,N-dichloro 2,2-dimethyl taurine at 4 mM and 40 mM significantly reduced bacterial counts and the presence of biofilm (visual interpretation from images). The higher concentration of N,N-dichloro 2,2-dimethyl taurine solution showed significantly more bacterial removal than the lower concentration.

Part C: Biofilm Prevention by N,N-dichloro 2,2-dimethyl taurine

Test Articles Used Were:
Sterile saline
White vinegar at 1:3 dilution with distilled water (filter sterilized).
Neomycin Prescription: 1 ml into 1000 ml of sterile saline
N,N-dichloro 2,2-dimethyl taurine, 40 mM, pH 4, 0.9% by weight NaCl

For Biofilm Prevention Study following sequential steps were taken:

Day 0: The test system, as described in detail above, utilized size 14 Foley catheters, cut and installed into a pre-sterilized flow system using aseptic techniques. Sterile medium was supplied to the system via a flow-break, to prevent back-growth into the medium reservoir. The entire system was placed into a 37° C. incubator. After conditioning the system with artificial urine medium for 30 minutes, each catheter was treated with a disinfectant. 20.0 ml of each treatment solution was loaded into 30 ml syringes and connected to a syringe pump. Sterile sections of tubing were attached from the syringe to the valve furthest from the flow break (bag end of the catheter). This end is designated as FRONT for sampling purposes. The pump was turned on and the treatments were introduced at 2.0 ml/min for 10 minutes through the catheters. Excess medium and treatment solution was captured in a waste container. After 10 minutes, the syringe pump was turned off and the solutions were left stationary in the catheters for 30 minutes. The solutions were then withdrawn back through the catheter into the syringe. The catheters were then rinsed with sterile medium for 30 minutes.

On Day 0 only: An inoculum from an overnight culture of urease positive Escherichia coli ATCC 25922 grown in artificial urine medium at 37° C. was introduced into the system via the valve closest to the flow break (bladder side of catheter). Each inoculum was tested for confirmation of urease production. After inoculation, the system remained under static conditions (no flow) for two hours, to allow for bacterial attachment to the catheters. Flow of artificial urine medium was then initiated and maintained at a rate of 0.75 ml/min.

Days 1, 3 and 5: For viable cell counts, a 3.0 centimeter section of tubing was removed and scraped with a sterile stainless steel rod (using aseptic technique) into a tube containing 10.0 ml of sterile PBS. The tubes were then sonicated for two minutes and the suspension was vortexed for one minute. The number of viable (culturable) bacteria was enumerated by serial dilution in PBS and plate counts using the spread-plate technique. Results are expressed as CFU/cm² and were calculated as described in Phase One.

Days 1 and 3: After sampling, the catheters were disinfected and rinsed with sterile medium as described above.

Days 2 and 4: the catheters were disinfected and rinsed with sterile medium as described above. No samples were taken.

Day 5: Samples were taken for imaging and both ends of the catheter were sampled for viable cell count data.

Results and Data Interpretations

TABLE 3
Biofilm prevention experiment.

| | Day | | | |
|---|---|---|---|---|
| | | | Day 5 | Day 5 |
| | | | “Front” | “End” |
| | Day 1 | Day 3 | (bag end) | (bladder end) |
| Saline | 7.6 | 7.3 | 7.7 | 8.0 |
| Vinegar | 5.7 | 3.4 | 4.1 | 3.9 |
| Neosporin | 5.5 | 3.2 | 3.8 | 4.5 |
| N,N-dichloro 2,2-dimethyl | 2.6 | 3.2 | 3.9 | 3.1 |
| taurine [40 mM] | | | | |

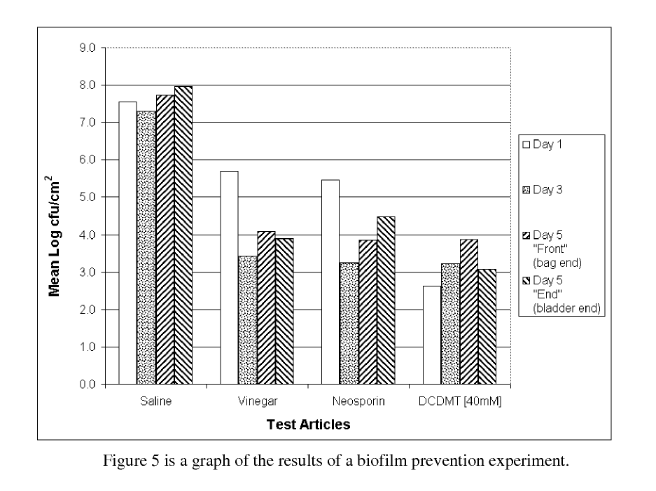

As seen in Table 3 and FIG. 5, N,N-dichloro 2,2-dimethyl taurine appeared to inhibit biofilm formation during the 5 day duration of this experiment. N,N-dichloro 2,2-dimethyl taurine appeared to be significantly better at inhibiting biofilm formation within the catheters compared to vinegar and Neosporin, especially by Day 5.

### EXAMPLE 7

### Establishes Reduction of Bacterial Count Using N,N-dichloro 2,2-dimethyl Taurine in a Catheter taken from a Patient

Test Articles Used Were:
Sterile phosphate-buffered saline (PBS)
N,N-dichloro 2,2-dimethyl taurine, 40 mM, pH 4, 0.9% by weight NaCl

Ex-vivo treatment of a patient catheter with N,N-dichloro 2,2-dimethyl taurine

A Foley catheter was removed from a patient by hospital personnel and placed in a sterile bag. In the Bozeman Deaconess Hospital (BDH) lab, the outside of the catheter was wiped down with 70% ethanol. Then the catheter was aseptically cut into 3 catheter portions (bag-end, middle and patient-end). Each portion was cut into 3.0 cm long sections using a ruler and razor blades.

Three of the sections (one bag-end, one middle and one patient-end) designated as control were placed into sterile PBS. Three of the sections (one bag-end, one middle and one patient-end) were placed in 40 mM N,N-dichloro 2,2-dimethyl taurine. All catheter sections were treated for 30 minutes individually in sterile glass tubes, each with sufficient solution to be immersed completely. After treatment, each 3 cm section was removed from the treatment tubes and the PBS control tubes and placed into a second glass tube containing sterile PBS for a 2 minute rinse in order to remove the treatment solution. The section was then removed from the tube and aseptically cut into 1.0 cm and 2.0 cm pieces. The 2.0 cm piece was placed in a tube containing 10 ml of sterile PBS, vortexed, sonicated and diluted for viable plate counts. The number of viable (culturable) bacteria was enumerated by serial dilution in PBS and plate counts using the spread-plate technique. Samples were plated on blood agar plates. Results will be expressed as colony-forming units/cm², CFU/cm² (calculated as 2.0 cm length×0.25 cm (radius)×3.14 (pi)=1.57 cm²). The 1.0 cm piece was placed in 4% formaldehyde solution.

Average Log CFU/cm² on catheter section after treatment

| | patient | | bag | average | Log |
|---|---|---|---|---|---|
| | end | middle | end | 3 pieces | reduction |
| PBS | 3.1 | 3.8 | 1.9 | 2.9 | 1.8 |
| N,N-dichloro | 0.8 | 0.5 | 2.1 | 1.2 | |
| 2,2-dimethyl taurine | | | | | |
| [40 mM] | | | | | |

Results

In average, treating catheter pieces with 40 mM N,N-dichloro 2,2-dimethyl taurine resulted in a 1.8 Log Reduction in bacterial growth compared to catheter pieces treated with sterile PBS.

### REFERENCES

Anwar, H., J. L. Strap, et al. (1992). “Eradication of biofilm cells of Staphylococcus aureus with tobramycin and cephalexin.” Can J Microbiol 38(7): 618-25.

Baillie, L. (1987). “Chlorhexidine resistance among bacteria isolated from urine of catheterized patients.” J Hosp Infect 10(1): 83-6.

Cho, Y. H., S. J. Lee, et al. (2001). “Prophylactic efficacy of a new gentamicin-releasing urethral catheter in short-term catheterized rabbits.” BJU Int 87(1): 104-9.

Costerton, J. W., P. S. Stewart, et al. (1999). “Bacterial biofilms: a common cause of persistent infections.” Science 284(5418): 1318-22.

Cravens, D. D. and S. Zweig (2000). “Urinary catheter management.” Am Fam Physician 61(2): 369-76.

Darouiche, R. O., J. A. Smith, Jr., et al. (1999). “Efficacy of antimicrobial-impregnated bladder catheters in reducing catheter-associated bacteriuria: a prospective, randomized, multicenter clinical trial.” Urology 54(6): 976-81.

Donlan, R. M. and J. W. Costerton (2002). “Biofilms: survival mechanisms of clinically relevant microorganisms.” Clin Microbiol Rev 15(2): 167-93.

Galloway, A. (1997). “Prevention of urinary tract infection in patients with spinal cord injury—a microbiological review.” Spinal Cord 35(4): 198-204.

Hashmi, S., E. Kelly, et al. (2003). “Urinary tract infection in surgical patients.” Am J Surg 186(1): 53-6.

Maki, D. G. and P. A. Tambyah (2001). “Engineering out the risk for infection with urinary catheters.” Emerg Infect Dis 7(2): 342-7.

Morris, N. S., D. J. Stickler, et al. (1999). “The development of bacterial biofilms on indwelling urethral catheters.” World J Urol 17(6): 345-50.

Muncie, H. L., Jr., J. M. Hoopes, et al. (1989). “Once-daily irrigation of long-term urethral catheters with normal saline. Lack of benefit.” Arch Intern Med 149(2): 441-3.

Pearman, J. W., M. Bailey, et al. (1991). “Bladder instillations of trisdine compared with catheter introducer for reduction of bacteriuria during intermittent catheterisation of patients with acute spinal cord trauma.” Br J Urol 67(5): 483-90.

Saint, S. and C. E. Chenoweth (2003). “Biofilms and catheter-associated urinary tract infections.” Infect Dis Clin North Am 17(2): 411-32.

Stickler, D., R. Young, et al. (2003). “Why are Foley catheters so vulnerable to encrustation and blockage by crystalline bacterial biofilm?” Urol Res 31(5): 306-11.

Stickler, D. J., G. L. Jones, et al. (2003). “Control of encrustation and blockage of Foley catheters.” Lancet 361(9367): 1435-7.

Tenke, P., C. R. Riedl, et al. (2004). “Bacterial biofilm formation on urologic devices and heparin coating as preventive strategy.” Int J Antimicrob Agents 23 Suppl 1: S67-74.

Trautner, B. W. and R. O. Darouiche (2004). “Catheter-associated infections: pathogenesis affects prevention.” Arch Intern Med 164(8): 842-50.

Trautner, B. W. and R. O. Darouiche (2004). “Role of biofilm in catheter-associated urinary tract infection.” Am J Infect Control 32(3): 177-83.

Trautner, B. W., R. A. Hull, et al. (2005). “Prevention of catheter-associated urinary tract infection.” Curr Opin Infect Dis 18(1): 37-41.

While the present invention is disclosed with reference to certain embodiments and examples as provided herein, these embodiments and examples are intended to be simply illustrative of the embodiments and examples, and are not intended to be limiting in scope. Accordingly, various modifications and variations will be apparent to one skilled in the art; and those modifications and variations fall within the scope of the invention and also fall within the claims below. All references, including patents, papers and texts cited in this application are incorporated by reference herein in their entirety.

1.-72. (canceled)

73. An aqueous antimicrobial composition for the treatment or prevention of a microbial infection in a patient, the composition comprising
(a) an antimicrobially effective amount of at least one N-halogenated amino acid, an N-halogenated amino acid derivative or an N-halogenated amino acid source;
(b) at least one halide salt selected from the group consisting of sodium chloride, sodium bromide, potassium chloride, potassium bromide, magnesium chloride, magnesium bromide and mixtures thereof; the halide salt concentration is from 0 to about 20 g/L of the aqueous composition;
(c) a pH of about 2 to about 8; and
(d) a constituent selected from the group consisting of calcium and magnesium chelating agents, biologically acceptable acids and/or salts thereof that are compatible with the antimicrobial composition, and mixtures thereof to maintain the pH at the range between about 2 and 8; and optionally
(e) an antimicrobially effective amount of HOBr or HOCl, or a source or composition capable of releasing HOBr or HOCl.

74. The composition of claim 73, wherein the biologically acceptable acid is citric acid and the biologically acceptable salt is potassium citrate.

75. The composition of claim 73, wherein the chelating agent is malic acid or maltol, and the biologically acceptable acid is citric acid.

76. The composition of claim 73 wherein the antimicrobially effective amount of the N-halogenated amino acid, the N-halogenated amino acid derivative or the N-halogenated amino acid source is about 0.1 mM to about 100 mM in the aqueous composition.

77. The composition of claim 73 wherein the pH is about 2 to about 5.

78. The composition of claim 73 wherein the N-halogenated amino acid comprises an N-halo- or N,N-dihaloamino acid of the formula (I))
A-C(R¹R^{o})R(CH₂)ₙ—C(YZ)—X′ (I)
wherein:
A is HalNH— or Hal₂N— wherein Hal is selected from the group consisting of chloro and bromo;
R is a carbon-carbon single bond or a divalent cycloalkylene radical with three to six carbon atoms;
R¹ is hydrogen, lower alkyl or the group —COOH;
R^{o} is lower alkyl;
n is 0 or an integer from 1 to 13, or
R¹ and R^{o} together with the carbon atom to which they attach form a (C₃-C₆)cycloalkyl ring;
Y is hydrogen, lower alkyl or —NH₂, —NHHal or —NHal₂;
Z is hydrogen or lower alkyl;
X′ is —COOH, —COOH or —P(═O)(OH)₂;
and if R is a divalent cycloalkylene radical, n is 11 or less;
wherein one hydrogen of the divalent cycloalkylene radical or in the divalent radical —(CH₂)ₙ— may be replaced with —NHHal or —NHal₂;
or a derivative thereof; or a derivative thereof, or a source of the N-halogenated amino acid of formula (I).

79. The composition of claim 73 wherein the N-halogenated amino acid comprises an N,N-dihalo-amino acid of the formula (II))
Hal₂N—C(R¹R^{o})—(CH₂)ₙ—C(YZ)—X (II)
wherein Hal is selected from the group consisting of chloro and bromo;
R¹ is lower alkyl or the group —COOH;
R¹ is lower alkyl;
or R¹ and R^{o} together with the carbon atom to which they attach form a (C₃-C₆)cycloalkyl ring;
n is 0 or an integer from 1 to 3;
Y is hydrogen, lower alkyl, —NH₂, —NHHal or —NHal₂;
Z is hydrogen or lower alkyl; and
X is —COOH or —SO₃H;
or a derivative thereof, or a source of an N-halogenated amino acid of formula (II).

80. The composition of claim 73 wherein the N-halogenated amino acid comprises a compound of the formula (IVA) or (IVB)
Hal₂N—C(R¹R²)—(CH₂)ₙ—C(YZ)—X (IVA)
HalNH—C(R¹R²)—(CH₂)ₙ—C(YZ)—X (IVB)
or a derivative thereof;
wherein Hal is selected from the group consisting of chloro and bromo;
R¹ is lower alkyl or the group —COOH;
R² is lower alkyl; or
R¹ and R² together with the carbon atom to which they attach form a (C₃-C₆)cycloalkyl ring;
n is 0 or an integer from 1 to 3;
Y is hydrogen, lower alkyl or —NH₂;
Z is hydrogen or lower alkyl; and
X is —COOH or —SO₃H;
wherein the derivative is selected from the group consisting of pharmaceutically acceptable salts, esters with lower alkanols, esters containing an aryl group, and wherein Y is C₁₋₆alkyl-CONH— or a source of an N-halogenated amino acid of formula (IVA) or (IVB).

81. The composition of claim 73 wherein the N-halogenated amino acid is a member selected from the group consisting of N,N-dichloro-2,2-dimethyltaurine, N-chloro-2,2-dimethyltaurine, N,N-dichloro-1,1,2,2-tetramethyltaurine, N-chloro-1,1,2,2-tetramethyl-taurine, N,N-dibromo-2,2-dimethyltaurine, N-bromo-2,2-dimethyltaurine, N,N-dibromo-1,1,2,2-tetramethyltaurine, N-bromo-1,1,2,2-tetramethyltaurine, N,N-dichloro-2-methyltaurine, N-chloro-2-methyltaurine, N,N-dichloro-2,2,3,3-tetramethyl-β-alanine, N-chloro-2,2,3,3-tetramethyl-β-alanine, N,N-dichloro-3,3-dimethylhomotaurine, N-chloro-3,3-dimethylhomotaurine, N,N-dichloro2-methyl-2-amino-ethanesulfonic acid, N-chloro-2-methyl-2-amino-ethanesulfonic acid, N,N-di-chloro-1-methyl-ethanesulfonic acid, N,N-dichloro-1-methyl-ethanesulfonic acid, N-chloroaminotrimethylene phosphonic acid, N,N-dibromo-2-amino-5-phosphono-pentanoic acid, N-bromo 2-amino-5-phosphonopentanoic acid, N,N-dichloro aminoethyl-phosphonic acid diesters, N,N-dichloro aminoethyl-phosphonic acid diethylester, N-chloro aminoethylphosphonic acid diesters, N-chloro aminoethylphosphonic acid diethylester, N,N-dichloro 1-amino-1-methylethane phosphonic acid, N-chloro 1-amino-1-methyl-ethane phosphonic acid, N,N-dichloro 1-amino-2-methylethane phosphonic acid, N-chloro 1-amino-2-methylethane phosphonic acid, N,N-dichloro 1-amino-2-methylpropane phosphonic acid, N-chloro 1-amino-2-methylpropane phosphonic acid, N,N-dichloro leucine phosphonic acid, N-chloro leucine phosphonic acid, N,N-dichloro-4-amino-4-phosphonobutyric acid, N-chloro 4-amino-4-phosphonobutyric acid, (±) N,N-dichloro 2-amino-5-phosphonovaleric acid, (±) N-chloro 2-amino-5-phosphono-valeric acid, N,N-dichloro (+)2-amino-5-phosphonovaleric acid, N-chloro (+)2-amino-5-phosphonovaleric acid, N,N-dichloro d,1-2-amino-3-phosphonopropionic acid, N-chloro d,1-2-amino-3-phosphonopropionic acid, N,N-dichloro 2-amino-8-phosphonooctanoic acid, N-chloro 2-amino-8-phosphonooctanoic acid, and a pharmaceutically acceptable salt or ester thereof.

82. The composition of claim 73 wherein the chelating agent concentration is selected to chelate up to about 10 mM of a member selected from the group consisting of calcium, magnesium and mixtures thereof.

83. The composition of claim 73 wherein the biologically acceptable acid and/or salt thereof concentration is about 1 mM to about 100 mM.

84. A method of treating a device for the prevention of bacteriuria or CAUTI or associated fungal or viral infections before using the device, the method comprising:
(a) contacting the device with an aqueous antimicrobial composition comprising;
an aqueous antimicrobial composition comprising;
(1) a composition comprising an antimicrobially effective amount of at least one N-halogenated amino acid, an N-halogenated amino acid derivative or an N-halogenated amino acid source or mixtures thereof; and optionally
(2) at least one halide salt selected from the group consisting of sodium chloride, sodium bromide, potassium chloride, potassium bromide, magnesium chloride, magnesium bromide and mixtures thereof;
the halide salt concentration ranging from 0.05 to about 20 g/L;
(3) a pH from about 2 to about 8; and optionally
(4) an antimicrobially effective amount of HOBr or HOCl, or a source or composition capable of releasing HOBr or HOCl; and
(5) a constituent selected from the group consisting of calcium and magnesium chelating agents, biologically acceptable acids and/or salts thereof that are compatible with the antimicrobial treatment system, and mixtures thereof, to maintain the pH at the range between about 2 and 8;
wherein contacting the device with the antimicrobial composition results in the prevention of blockage of the device by biofilm and/or encrustation.

85. The method of claim 84, wherein the device is a catheter.

86. The method of claim 84 wherein the antimicrobially effective amount of the N-halogenated amino acid, or the N-halogenated amino acid derivative or the source or composition capable of releasing HOBr or HOCl is about 2 mM to about 50 mM.

87. The method of claim 84, wherein the halide salt concentration is about 0.1 to about 10 g/L.

88. The method of claim 84 wherein the biologically acceptable acid is a member selected from the group consisting of acetic acid, benzoic acid, citric acid, propionic acid, oxalic acid, hydrochloric acid, phosphoric acid, sulfuric acid, boric acid, diethylenetriamine pentaacetic acid, and esters of p-hydroxybenzoic acid (Parabens), or the biologically acceptable salt form of the acid is selected from the group consisting of potassium citrate, potassium metaphosphate, sodium acetate, and sodium phosphate.

89. The method of claim 84 wherein the total antimicrobially effective amount of the N-halogenated amino acid, the N-halogenated amino acid derivative, or the N-halogenated amino acid source and HOBr or HOCl, or the source of HOBr or HOCl is from about 2 mM to about 20 mM.

90. A method of treating, inhibiting or preventing a microbial infection in or near a medical device before or after the device has been inserted into a patient comprises the following treatment steps in isolation or in combination, and using the following composition:
(A) an antimicrobially effective amount of at least one N-halogenated amino acid, or an N-halogenated amino acid derivative or an N-halogenated amino acid source; and optionally
(B) at least one halide salt selected from the group consisting of sodium chloride, sodium bromide, potassium chloride, potassium bromide, magnesium chloride, magnesium bromide and mixtures thereof, the halide salt concentration ranging from 0.05 to about 20 g/L of the composition;
(C) wherein the pH of the composition is about 2 to about 8; and
(D) wherein the antimicrobially effective amount of the N-halogenated amino acid, or the N-halogenated amino acid derivative or the N-halogenated amino acid source is about 1 mM to about 1000 mM of the composition; and optionally
(E) an antimicrobially effective amount of HOBr or HOCl, or a source or composition capable of releasing HOBr or HOCl; and
(F) a constituent selected from the group consisting of calcium and magnesium chelating agents, biologically acceptable acids and salts thereof that are compatible with the antimicrobial treatment system, and mixtures thereof to maintain the pH between about 2 and 6 and to prevent blockage of the catheter by biofilm and/or encrustation;
(a) contacting the device with a composition comprising elements (A) through (D), and optionally elements (E) or (F), prior to insertion of the device into a patient or after removal of the device from the patient;
(b) washing, bathing or flushing the device with a composition comprising elements (A) through (D), and optionally elements (E) or (F), prior to insertion of the device into a patient or after removal from the patient;
(c) irrigating the device with a composition comprising elements (A) through (D), and optionally elements (E) or (F), after insertion into the patient, to remove encrustations on the device; or
(d) instilling through the device a composition comprising elements (A) through (D), and optionally elements (E) or (F), into the patient to treat or prevent a fungal or bacterial infection.

91. The method of claim 90, wherein the insertion of the device is into the bladder of a patient and the method is used to treat or prevent a fungal or bacterial infection of the lining of the bladder.

92. The method of claim 90, wherein the chelating agent is malic acid or maltol, and the biologically acceptable acid is citric acid.
